(19) United States
(12) Patent Application Publication
(10) Pub. No.: US 2011/0022057 A1
(43) Pub. Date: Jan. 27, 2011

(54) APPARATUS AND METHODS FOR TRANSFERRING AN IMPLANTED ELONGATE BODY TO A REMOTE SITE

(75) Inventors: Neal L. Eigler, Malibu, CA (US); James S. Whiting, Los Angeles, CA (US); John L. Wardle, San Clemente, CA (US); Werner Hafelfinger, Thousand Oaks, CA (US); Apratim Dixit, Burbank, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/842,917

(22) Filed: Jul. 23, 2010

Related U.S. Application Data
(63) Continuation-in-part of application No. 11/622,654, filed on Jan. 12, 2007.
(60) Provisional application No. 60/764,878, filed on Feb. 3, 2006.

(51) Int. Cl. A61B 19/00

(52) U.S. Cl. 606/129

(57) ABSTRACT

A transfer guidewire assembly configured to manipulate an implanted elongate body includes a flexible elongate portion, such as a guidewire, and coupler. The flexible elongate body has a proximal end and a distal end attached to the coupler. The coupler can include a catheter and/or a handle. The handle can include a screw. The coupler is configured to be removably attached to the end of an implanted elongate body, for example, by forming an interference fit with the outside diameter of the implanted body. A method for transferring an end of an implanted medical component from first site to a second site within a patient, such as a pacemaker, defibrillator, and/or sensor lead, etc., includes inserting a guidewire into the body at the first site and externalizing the guidewire at the second site. A proximal portion of the implanted component near the first site and is attached to the guidewire. The proximal portion of the implanted component is pulled through the patient's body and out the second site with the transfer guidewire assembly.

### CROSS-REFERENCE TO RELATED APPLICATIONS

The present application is a continuation-in-part of U.S. application Ser. No. 11/622,654, filed on Jan. 12, 2007, which claims priority under 35 U.S.C. §119(e) to U.S. Provisional Application No. 60/764,878 filed on Feb. 3, 2006, the disclosures of which are herein incorporated by reference in their entirety.

### FIELD OF THE INVENTION

This invention generally relates to devices, systems and methods for transferring a device from a first location, such as an initial insertion site on the body, to a second location, such as a different insertion site on the body.

### BACKGROUND OF THE INVENTION

The placement of a permanently or temporarily implantable device in the left side of the heart, and particularly the left atrium, may be difficult at a particular site of insertion because an operator must contend with the anatomical obstacles or equipment limitations presented by the catheter's route to the left heart. For example, it is more difficult to access the left atrium by performing an atrial transseptal puncture from an insertion point on the neck or near the shoulder than it is to perform a standard transfemoral Brockenbrough needle puncture of the intra-atrial septum from the right groin region. Because of the rigidity of the Brockenbrough catheter/needle system, the insertion site must provide a relatively straight path to the intra-atrial septum. A superior insertion site, however, provides a significantly tortuous and winding pathway to the intra-atrial septum, which makes the use of a Brockenbrough needle puncture technically more difficult from this insertion site. Still, there may be advantages to performing a medical procedure through a certain route that is difficult to catheterize. For example, it can be difficult to perform mitral balloon valvuloplasty from the inferior venous approach because an abrupt curve must be made in the left atrium to reach the mitral valve. When a valvuloplasty balloon is passed from a superior venous approach through the intra-atrial septum, there is a generally straight pathway to the mitral valve. Likewise, the implantation of certain medical devices may benefit from implantation through routes that are difficult to catheterize. One example is a medical device as described in U.S. Pat. No. 6,328,699, herein incorporated by reference, whereby a pressure transducer is placed on the left atrial side of the intra-atrial septum using transseptal catheterization. In some embodiments of the '699 patent, the pressure transducer is in continuity with a lead to a proximal housing that is more convenient when implanted in the subcutaneous tissue near the shoulder. Thus, although the catheterization is more readily performed from the groin region, insertion of the implanted device from the shoulder is preferred.

### SUMMARY

Several embodiments of the current invention provide a new method that allows transseptal catheterization of the left atrium from the standard transfemoral route via the groin that places the distal end of a guidewire in the vicinity of the left atrium followed by transfer of the proximal end from the groin to exit from a superior vein (subclavian or jugular).

In one embodiment, a method for transferring an implanted medical component (such as a guidewire) from an initial insertion site of the vasculature to an exit site of the vasculature in a patient with pre-existing implanted device, such as a pacemaker, defibrillator or diagnostic sensor system, is provided. In one embodiment, a guidewire is inserted into the vasculature at an insertion site and externalized at a separate exit site of the vasculature. Guidewire insertion is performed using a protective barrier (such as a sheath) having one or more ports or lumena to, for example, reduce the risk of entanglement with the pre-existing implanted device.

In one embodiment, the implantable component is inserted from the insertion site and anchored to its target location, such as the left atrium or left ventricle. The proximal portion of the implanted component proximate to the insertion site is connected to the pull member and pulled through the vasculature and out the exit site. A coupling device may be used to facilitate attachment of the pull member and vasculature. The pull member may be connected to the implanted component before or after the insertion of the implantable component to the target location. The insertion and exit sites may be external sites, such as the skin, or internal sites, such as a wall of a blood vessel.

In a further embodiment, the protective sheath is further configured or provided in conjunction with a puncturing assembly for penetrating through various tissue structures to facilitate access to the target location. In some embodiments, the puncturing assembly is located at the distal end of the protective sheath, but in other embodiments, the puncturing assembly is located anywhere between the proximal end and distal end of the sheath.

In some embodiment, a guide wire or lead with a puncturing or sharpened tip is provided in addition to or instead of the puncturing assembling.

In some embodiments, a method for positioning the distal end of an implantable lead in a target site and providing access to the proximal end of the implantable lead at a site different than the insertion site in a patient is provided, comprising providing a sheath, a pull wire, a guidewire, and an implantable lead, wherein said sheath has a proximal end, a body, and a distal end, wherein said guidewire has a proximal end, a body, and a distal end, wherein said transfer has a proximal end, a body, and a distal end, and wherein said implantable lead has a proximal end, a body, and a distal end; inserting the sheath into the vasculature of a patient at an insertion site; passing the distal end of the pull wire through the distal end of the sheath and toward a second access site of said vasculature while maintaining at least a portion of the pull wire body within the vasculature; passing the distal end of the guidewire through the sheath and through the side port of the sheath body to a target site; removing the sheath from the vasculature; passing the implantable lead to the target site using the guidewire; coupling the proximal end of the pull wire to a proximal end of the implantable lead; and externalizing the proximal end of the implantable lead out of said vasculature, thereby providing access to the proximal end of the implantable lead at an exit site different than the insertion site.

The sheath may comprise a tissue penetration member configured to extend from the side port. The method may further comprise penetrating a body tissue structure with the tissue penetration member to provide a tissue pathway to the target site. The method may further comprise using a snare to pull the distal end of the pull wire out of the vasculature. The method may further comprise piercing through a body structure with a penetration member. Externalizing the proximal end of the implantable lead out of said vasculature may comprise removing the proximal end of the implantable lead from the body of the patient. Coupling may comprise using a connector to connect the proximal end of the pull wire with the proximal end of the implantable lead. The pull wire includes, but is not limited to a guidewire, a snare, and a suture. The implantable lead includes, but is not limited to a pressure sensor lead or a pacemaker/defibrillator lead. The method target site may be selected from a group consisting of the left atrium, the left ventricle, the right atrium, the right ventricle, a pulmonary artery, the coronary sinus, and the left atrial appendage. In some embodiments, at least one of the first and second access sites may be selected from a group consisting of a jugular vein or carotid artery or their branch vessels, a subclavian blood vessel, an axillary blood vessel, a femoral blood vessel, an iliac blood vessel, a brachiocephalic vein, a superior vena cava and an inferior vena cava, a right atrial wall, a left atrial wall, a left ventricular wall, a left ventricle apex, a right ventricular wall or outflow tract or apex.

In several embodiments, a method for placing an elongate member in a body space is provided. Suitable body spaces include, but are not limited to, blood vessels, heart chambers, the spinal canal, the nasopharynx, the oropharynx, the hypopharynx, the esophagus, the biliary tract, the stomach, small intestine, large intestine, rectum, the genitourinary tract, the bronchial tree, etc.

In one embodiment, the protective sheath comprises a tubular conduit that is inserted into a body space at an insertion site, such as at an incision site, orifice, duct, or other opening. Tubular conduits include, but are not limited to catheters, introducers and sheaths, sleeve or other coverings. The distal end of a first elongate member is inserted into the tubular conduit and externalized from the body space at an exit site. Elongate members include, but are not limited to, guidewires, snares or suture lines. The distal end of a second elongate member is then inserted into the conduit and positioned at a target site accessible from the body space. The proximal end of the first elongate member is attached to a proximal end of the second elongate member. The proximal end of the second elongate member is externalized from the exit site of the body lumen.

In another embodiment, a method for placing an elongate member in a body space is provided, comprising inserting a tubular conduit into a body space at an insertion site, inserting a distal end of a first elongate member into the tubular conduit, externalizing the distal end of the first elongate member from the body space at an exit site, inserting a distal end of a second elongate member into the conduit, positioning the distal end of the second elongate member at a target site accessible from the body space, attaching a proximal end of the first elongate member to a proximal end of the second elongate member, and moving the proximal end of the second elongate member toward the exit site of the body space. The body space may be a lumen of the cardiovascular system. The body space may contain a pre-existing implant at least partially positioned in the body space between the insertion site and the exit site. The pre-existing implant may be positioned in the body cavity prior to externalizing the proximal end of the second elongate member. Inserting the distal end of the second elongate member into the conduit may occur before externalizing the distal end of the first elongate member from the body space at an exit site. The method may further comprise passing the distal end of the second elongate member through a side opening of the tubular conduit. Attaching the proximal end of the first elongate member to the proximal end of the second elongate member may comprise attaching the proximal end of the first elongate member to a first end of a coupler and attaching proximal end of the second elongate member to a second end of the coupler. The method may further comprise passing the distal end of a second elongate member out of a middle aperture of the tubular conduit. The method may further comprise passing a puncture structure out of the middle aperture of the tubular conduit. The method may further comprise puncturing a through a tissue with the puncture structure to access the target site. Inserting the distal end of the first elongate member into the tubular conduit may comprise inserting the distal end of the first elongate member into a common lumen of the tubular conduit, and wherein inserting the distal end of the second elongate member into the tubular conduit may comprise inserting the distal end of the second elongate member into the common lumen of the tubular conduit.

In one embodiment, a method for manipulating an implanted component is provided, comprising accessing a body space of a body, the body space containing at least one pre-existing foreign body, placing a protective barrier into the body space, wherein the protective barrier comprises a protected pathway and a wall separating the protected pathway from the at least one pre-existing foreign body, placing an elongate member through the protected pathway, such that the elongate member has a first end and a second end located outside the body space, inserting at least a portion of an implantable component through at least a portion of the protected pathway and into the body, such that the implantable component has a first end located outside the body and a second end located at a target site in the body, and connecting the first end of the elongate member to the first end of the implantable component. The method may further comprise removing the protective barrier from the body space, which, in some embodiments, may be performed before connecting the first end of the elongate member to the first end of the implantable component.

In one embodiment, a device for manipulating an implantable lead member is provided, comprising a biocompatible elongate member having a first section and a second section, wherein the first section comprises a pull member attachment interface and the second section comprises a selective coupling interface configured to selectively attach to a section of an implantable lead member. The pull member interface may be a guidewire attachment interface or snare attachment interface. The pull member interface may comprise a cavity configured to accept the pull member, a collet structure about the cavity, and a locking member configured to selectively compress the collet structure. The selective coupling interface may comprise a cavity configured to accept the implantable lead member, a collet structure about the cavity, and a locking member configured to selectively compress the collet structure. The biocompatible elongate member may comprise a flexible or rigid material.

In some embodiments, a kit, system or compilation of materials for manipulating an implantable lead member is provided, comprising a biocompatible elongate member having a first section and a second section, wherein the first section comprises a pull member attachment interface and the second section comprises a selective coupling interface configured to selectively attach to a section of an implantable lead member. The kit may further comprise a pull member, snare and/or a transfer guidewire. The kit may further comprise a sheath having a proximal end, a distal end, and a first lumen comprising a first section extending at least between the proximal end and a side opening between the proximal end and the distal end. The kit may further comprise a tissue wall puncture member configured to movably reside at least within the puncture lumen. The first lumen comprises a second section between the side opening and the distal end of the sheath. The sheath may further comprise a second lumen between the proximal end and the distal end of the sheath, wherein the second lumen is separate from the first lumen. The kit may further comprise at least one vascular sheath, and in some embodiments comprises two vascular sheaths. The vascular sheaths may have different dimensions. The kit may also comprise instructions for using one or more kit components. In one embodiment, the kit further comprises instructions for using the biocompatible elongate member with a pull member and the implantable lead member.

In one embodiment, a method of transferring a guidewire from one insertion site to another insertion site is provided. In one embodiment, the method comprises the steps of introducing a first guidewire to a first insertion site, wherein the first guidewire has a proximal and distal end, introducing the distal end of the first guidewire to a target site, introducing a catheter having a proximal end and a distal end from a second insertion site and advancing the distal end of the catheter to the proximity of the first insertion site, introducing a second guidewire, wherein the second guidewire has a proximal and a distal end, through the catheter such that the distal end of the second guidewire extends out through the first insertion site, advancing the catheter over the second guidewire whereby a portion of the catheter emerges from the body through the first insertion site, and removing the second guidewire entirely from the catheter and inserting the proximal end of the first guidewire into the distal end of the catheter, whereby the proximal end of the first guidewire exist the proximal end of the catheter at the second insertion site. This method may further comprise snaring of distal end of the second guidewire with a snare and pulling the snare and the distal end of the second guidewire out from the first insertion site. In some embodiments of the invention, an introducer is placed at the first insertion site and/or second insertion site. In some embodiments, the introduction of the distal end of the first guidewire to a target site comprises introducing the distal end of the first guidewire to a site in the left atrium, right ventricle, pulmonary artery or renal artery. In some embodiments, the introduction of the catheter over the second guidewire from the second insertion site to the first insertion site comprises introducing a catheter from the second insertion site to a right femoral vein or right common carotid artery, or from a left femoral vein or left axillary vein to the first insertion site.

In one embodiment, another method of transferring a guidewire from one insertion site to another insertion site using a second guidewire is provided. In one embodiment, the method comprises the steps of introducing a first guidewire to a first insertion site, wherein the guidewire has a proximal end and a distal end, introducing the distal end of the first guidewire to a target site, introducing a catheter having a proximal end and a distal end from a second insertion site and advancing the distal end to the proximity of the first insertion site, introducing a second guidewire, wherein the second guidewire has a proximal end and a distal end, through the catheter such that the distal end of the second catheter extends out through the first insertion site, advancing the catheter over the second guidewire whereby a portion of the catheter emerges from the body through the first insertion site, engaging the proximal end of the first guidewire to the distal end of the second guidewire and withdrawing the catheter, second guidewire and the proximal end of the first guidewire from the second insertion site.

In one embodiment, another method of transferring a guidewire from one insertion site to another insertion site is provided, comprising the steps of introducing a guidewire through a first insertion site, introducing a catheter through a second insertion site to the first insertion site and inserting the proximal end of the guidewire into the distal end of the catheter whereby the proximal end of the guidewire exits the proximal end of the catheter at the second insertion site. In a further embodiment, the guidewire is introduced to a target site when the guidewire is introduced through the first insertion site. In another embodiment, when introducing the distal end of the catheter through a second insertion site to the first insertion site, the distal end of the catheter exits from the first insertion site.

In another embodiment, a method of transferring a guidewire from one insertion site to another insertion site using a conduit is provided. In one embodiment, the method comprises the steps of introducing the distal end of a guidewire through a first insertion site, establishing access to a second insertion site, introducing a conduit between the first insertion site and the second insertion site, where the conduit has a first end at the first insertion site and a second end at the second insertion site, inserting the proximal end of the guidewire into the first end of the conduit whereby the proximal end of the guidewire exists the second end of the conduit. In further embodiments of the invention, the conduit is a catheter. In still further embodiments, the step of introducing the conduit between the first insertion site and the second insertion site comprises introducing the catheter from the second insertion site to the first insertion site.

In another embodiment, another method of transferring a guidewire is provided, comprising the steps of providing a guidewire having a proximal end and a distal end, passing the proximal end and the distal end of the guidewire through a first insertion site in the body, where the distal end is passed before the proximal end, and externalizing the proximal end through a second insertion site of the body while the distal end remains in the body. This method may further comprise the step of passing a medical device over the guidewire into the body. The medical device may be a therapeutic or diagnostic medical device. The passing step may also involve a transseptal puncture. The externalizing step may involve inserting a snare through the second insertion site to engage the proximal end of the guidewire with the snare and withdrawing the snare and the proximal end of the guidewire from the second insertion site. One example of the first insertion site is the femoral vein, while one example of the second insertion site includes the subclavian vein.

In another embodiment, another method of transferring a guidewire from a first insertion site to another insertion site is provided. In one embodiment, the method comprises the steps of providing a guidewire with a proximal end, middle segment and a distal end, passing the proximal end and the distal end of the guidewire through a first insertion site into the body, wherein the distal end of the guidewire is passed before the proximal end of the guidewire and at least some portion of the middle segment remains external to the first insertion site, externalizing the proximal end of the guidewire through a second insertion site of the body while the distal end of the guidewire remains in the body and drawing the external portion of the middle segment into the body through the first insertion site. The method may further comprise the step of maintaining at least a portion of the middle segment of the guidewire outside the body while the proximal end and the distal end are inside the body.

In another embodiment, a method of transferring a guidewire from one insertion site to another is provided, comprising the steps of providing a guidewire having a proximal end and a distal end, inserting the distal end through a first insertion site of a body and through a pivot point in the body, inserting the proximal end through the first insertion site and externalizing the proximal end through a second insertion site without passing the proximal end through the pivot point.

In still another embodiment of the invention, a method of transferring a guidewire from one insertion site to another insertion site is provided. In one embodiment, the method comprises the steps of providing a guidewire having a proximal end and a distal end, passing the distal end the guidewire from a first insertion site in a body to a target site in the body, passing the proximal end of the guidewire from the first insertion site to a second insertion site, where the proximal end does not enter the target site when passing to the second insertion site. The method may further comprise the steps of providing a medical device and passing at least a portion the medical device along the guidewire from the second insertion site to the target site. The medical device may be a therapeutic or diagnostic medical device. One example of the first insertion site is a femoral vein, while one example of the second insertion site is a subclavian vein.

In one embodiment, a method of inserting a pacemaker lead through a sheath to the proximity of the left atrium is provided. In one embodiment, the method comprises the steps of providing a guidewire having a proximal end and a distal end, defining a first pathway from the right femoral vein to the left atrium through the right atrium, defining a second pathway from the right femoral vein to a subclavian vein through the right atrium; wherein the second pathway does not traverse the left atrium, defining a third pathway from the subclavian vein to the left atrium through the right atrium, passing the distal end of the guidewire along the first pathway, passing the proximal end of the guidewire along the second pathway, providing a sheath for passing a pacemaker lead, passing the sheath over the guidewire along the third pathway, withdrawing the guidewire from the sheath, providing a pacemaker lead and passing the pacemaker lead through the sheath along the third pathway, thereby inserting the pacemaker lead into the left atrium.

In other embodiments, a method of transferring a guidewire from one insertion site to another insertion site is provided. In one embodiment, the method comprises the steps of providing a guidewire having a proximal end and a distal end, defining a first pathway in a body from a first insertion site on a body to a target area in the body, defining a second pathway from the first insertion site to a second insertion site on the body, wherein the second pathway does not traverse the target area, defining a third pathway from the second insertion site to the target area, passing the distal end along the first pathway and passing the proximal end along the second pathway. The method may further comprise the steps of providing a medical device and passing at least a portion of the medical device along the third pathway. In further embodiments, the first pathway crosses the intra-atrial septum. In other embodiments, the first, second and third pathways each pass through a junction area such as the right atrium. The medical device can be a therapeutic and/or diagnostic medical device. One example of the first insertion site is the femoral vein, while one example of the second insertion site includes the subclavian vein.

In another embodiment, a method of transferring a medical device component from one insertion site to another insertion site is provided. In one embodiment, the method comprises the steps of introducing a medical device component to a first insertion site, wherein the component has a proximal end and a distal end, introducing a guidewire to a second insertion site, wherein the guidewire has a proximal end and a distal end, introducing the distal end of the medical device component to a target site, introducing a catheter having a proximal end and a distal end over the second guidewire from the second insertion site to the first insertion site, wherein the distal end of the catheter exits the first insertion site, and inserting the proximal end of the medical device component into the distal end of the catheter whereby the proximal end of the medical device component exits the proximal end of the catheter at the second insertion site. Medical devices in this and other embodiments include, but are not limited to, clinical, diagnostic and therapeutic devices. Therapeutic devices include, but are not limited to, drug delivery devices, radiation agents, brachytherapy agents, pacemakers, defibrillators, valves, stents, sensors and pumps, and combinations thereof.

In another embodiment, a method of transferring a medical device component from one insertion site to another insertion site is provided. In one embodiment, the method comprises the steps of introducing the distal end of a medical device component through a first insertion site, wherein the component has a proximal end and a distal end, removably engaging the distal end of an extension device to the proximal end of the medical device component, wherein the extension device has a proximal end and a distal end, advancing the distal end of the medical device component to a target site, introducing a guidewire to a second insertion site, wherein the guidewire has a proximal end and a distal end, introducing a catheter having a proximal end and a distal end over the second guidewire from said second insertion site to said first insertion site, wherein the distal end of said catheter exits said first insertion site, inserting the proximal end of the extension device into the distal end of the catheter whereby the proximal end of the extension device exits the proximal end of the catheter at the second insertion site, and withdrawing the catheter and the extension device from the second insertion site whereby the proximal end of the medical device component is externalized through the second insertion site. In a further embodiment of the invention, in the step of advancing the medical device component to the target site, the proximal end of the extension device remains outside the body at the first insertion site when the medical device component is advanced entirely inside the body. The embodiment may also comprise the steps of snaring the distal end of the guidewire with a snare from the first insertion site and pulling the snare and the distal end of the second guidewire from the first insertion site. An introducer may also be placed at the first and/or the second introducer site. The target sites may comprise in the left atrium, right ventricle, pulmonary artery and coronary sinus. The first insertion sites may comprise the right femoral vein and right carotid artery. The second insertion sites may comprise the left femoral vein and the left axillary artery. The medical device component may comprise a second guidewire, an implantable sensor lead, or a temporary sensor lead.

Another embodiment provides a method of transferring a pacemaker lead from the right femoral vein to the right subclavian vein, comprising the steps of introducing the distal end of a pacemaker lead having a proximal end and a distal end through the right femoral vein, introducing the distal end of a catheter having a proximal end and a distal end through the right femoral vein and advancing the proximal end of the catheter to exit from the right subclavian vein, and inserting the proximal end of the pacemaker lead into the proximal end of the catheter whereby the proximal end of the pacemaker lead exits the distal end of the catheter at the right subclavian vein.

Another embodiment provides a method of transferring a medical device component from one insertion site to another insertion site, comprising the steps of introducing the distal end of a medical device component having a proximal end and a distal end through a first insertion site, introducing the distal end of a catheter having a proximal end and a distal end through the first insertion site and adjacent to a second insertion site, and inserting the proximal end of the medical device component into the proximal end of the catheter whereby the proximal end of the medical device component exits the distal end of the catheter at said second insertion site. The medical device component could be a pacemaker lead. One example of the first insertion site is the right femoral vein, while the second insertion site may be selected from the group consisting of one or more of the following, including the right subclavian vein, left subclavian vein, right jugular vein and left jugular vein.

In another embodiment, a method of transferring a medical device component from one insertion site to another insertion site is provided. In one embodiment, this method comprises the steps of providing a medical device component having a proximal end and a distal end, passing both the proximal end and the distal end of the medical device component through a first insertion site into a body, wherein the distal end is passed before the proximal end, externalizing the proximal end through a second insertion site of the body while the distal end remains in the body.

Another embodiment provides a method of transferring a medical device component from one insertion site to another insertion site. In one embodiment, this method comprises providing a medical device component having a proximal end and a distal end, passing both the proximal end and the distal end of the medical device component through a first insertion site into a body, wherein the distal end is passed before the proximal end, and externalizing the proximal end of the medical device component through a second insertion site of the body while the distal end remains in the body.

In another embodiment, a method of transferring a medical device component from one insertion site to another insertion site is provided. In one embodiment, this method comprises providing a medical device component having a proximal end and a distal end, passing the distal end of said medical device from a first insertion site of a body to a target site in the body; and passing the proximal end of the medical device through the body from the first insertion site to a second insertion site, wherein the proximal end does not enter the target site when passing to the second insertion site. Furthermore, the step of passing the proximal end of the medical device component of the comprises passing a snare from the second insertion site to the first insertion site, snaring the proximal end of the medical device component with the snare and withdrawing the snare and the medical device component from the second insertion site. One example of the medical device component is a pacing lead of a cardiac pacemaker. One example of the target site is the coronary sinus.

In another embodiment, a method of manipulating a device insertion pathway from one insertion site to another insertion site is provided. In one embodiment, this method comprises providing an insertion pathway between a first insertion site and a target site in the body, wherein the insertion pathway comprises a proximal segment, a distal segment and a pivot point between the proximal segment and the distal segment; and manipulating the proximal segment by pivoting the proximal segment at the pivot point from the first insertion site to a second insertion site, wherein the proximal segment does not overlap the distal segment.

In one embodiment, a kit for performing a transfer of a guidewire from one insertion site to another insertion site is provided. In one embodiment, the kit, system, collection, or combination of materials, comprises at least two guidewires and a catheter. The kit may also comprise a snare, at least one introducer and/or a Brockenbrough needle catheter. In some embodiments of the kit, at least one guidewire comprises a movable inner core mandrel.

In another embodiment, a guidewire for manipulating the insertion pathways to target sites in the body is provided. In one embodiment, this guidewire comprises a guidewire body with a proximal end, distal end and a middle segment, and an internal lumen comprising a movable core mandrel. The mandrel is operable to be inserted into the internal lumen during guidewire insertion and extracted from the internal lumen during guidewire transfer. The guidewire is at least about 180 cm in length. In further embodiments of the guidewire, the guidewire has a length of about 240 cm. In other embodiments of the guidewire, the internal lumen extends substantially through the length of the guidewire. In still other embodiments of the guidewire, the distal end of the guidewire is capable of a first configuration when the mandrel is in a retracted position and a second configuration when the mandrel is in an extended position. In some embodiments, the first configuration is a spiral coiled configuration or a J-shaped configuration. In some embodiments, the second configuration is a straight configuration or angled configuration.

In another embodiment, a guidewire with adjustable flexibility is provided. In one embodiment, this guidewire comprises a first component having a proximal end, a distal end and an elongate flexible body extending therebetween, and a second component, axially movably associated with the first component, the second component having a proximal end, a distal end and an elongate flexible body extending therebetween. The axial movement of one of the first and second components with respect to the other of the first and second components changes the lateral flexibility of the guidewire. At least one component of the guidewire has a length of at least about 180 cm. The first component may comprise a tube or a core. In some embodiments, the second component has an axial length within the range of about 20% to about 200% of the axial length of the first component. In other embodiments, the second component has an axial length of about 110% of the axial length of the first component. In still other embodiments, the guidewire is dimensioned to percutaneously enter and translumenally navigate a lumen for directing at least a component of a medical device to a remote target site.

In another embodiment, another guidewire with adjustable flexibility is provided. This guidewire comprises an elongate flexible tubular body having a proximal end and a distal end, a central lumen extending distally into the tubular body from the proximal end, and an elongate flexible core wire axially moveable within the central lumen. Axial proximal retraction of the core wire with respect to the tubular body increases the flexibility of at least a portion of the guidewire, and axial distal advance of the core wire with respect to the tubular body decreases the flexibility of at least a portion of the guidewire. The length of the elongate flexible tubular body is at least about 180 cm. In some embodiments, the portions of the guidewire capable of changes in flexibility define a flexibility zone of the guidewire. In some embodiments, the flexibility zone comprises at least about the proximal 90% length of the elongate tubular body. In other embodiments, the flexibility zone comprises generally the entire length of the elongate tubular body.

In another embodiment, another method of treating a patient is provided, comprising the steps of introducing a guidewire through a first access site into the patient's body, advancing the guidewire translumenally to a target site, adjusting the flexibility of the guidewire, and moving at least a portion of the guidewire to a second access site. In some embodiments, the step of adjusting the flexibility of the guidewire comprises distally advancing a core wire within the guidewire, while in other embodiments, it comprises distally advancing a tubular support around the outside of the guidewire.

In still another embodiment, a method of accessing a target site is provided. In one embodiment, this method comprises introducing a guidewire into a patient through an introduction site, the guidewire having a first, reduced flexibility, externalizing at least a portion of the guidewire through a different site of the body, and adjusting the guidewire to have a second flexibility. In further embodiments, the method also comprises the step of introducing a catheter along the guidewire after adjusting the guidewire to have a second flexibility.

In yet another embodiment, a transfer guidewire assembly is configured to manipulate an implanted elongate body. Transfer guidewire assembly includes a flexible elongate body and a coupler. The flexible elongate body has a proximal end and a distal end. The coupler is attached to the flexible elongate body's distal end. The coupler is configured to be removably attached to the end of an implanted elongate body.

In one embodiment, the flexible elongate body includes a guidewire. In another embodiment, the coupler includes a screw. In another embodiment, the coupler is configured to rotate about the flexible elongate body. In another embodiment, the coupler is configured to prevent rotational forces from acting upon the implanted elongate body as the flexible elongate body is withdrawn from the patient's body while attached to the implanted elongate body. In another embodiment, the transfer guidewire assembly also includes a stylet extending from the distal end of the flexible elongate body. In another embodiment, the coupler includes a rotational coupling. In yet another embodiment, the implanted elongate body comprises a sensor lead.

In another embodiment, a transfer guidewire assembly is configured to reposition an end of an implanted lead from a first access point at a patient's body to a second access point at the patient's body. In one embodiment, the transfer guidewire assembly includes a flexible guidewire and a rotational coupling. The flexible guidewire has a proximal end and a distal end, and the rotational coupling is attached to the guidewire's distal end. The rotational coupling is configured to removably attach to a lead implanted in a patient's body.

In one embodiment, the rotational coupling includes a housing having an atraumatic surface configured to be pulled through the patient's body from a first access point to a second access point while attached to the implanted lead without damaging tissue within the patient's body In another embodiment, the rotational coupling includes a screw configured to mate with the implanted lead. In another embodiment, the transfer guidewire assembly also includes a stylet extending from a distal end of the rotational coupling that is sized to enter the implanted lead.

In another embodiment, a transfer guidewire assembly is configured to reposition an end of an implantable, flexible, elongate body from a first access point at a patient's body to a second access point at the patient's body. The transfer guidewire assembly includes a flexible guidewire and a catheter. The flexible guidewire has a proximal end and a distal end. The catheter is attached to the guidewire's proximal end. The catheter is configured to form an interference fit over an end of a flexible, elongate body implantable within a medical patient.

In one embodiment, the distal end of the flexible guidewire is retrievable with a snare. In another embodiment, the distal end of the flexible guidewire includes a J-tip. In another embodiment, the catheter is configured to form an interference fit over an end of the flexible, elongate body. For example, in one embodiment, the interference fit allows the catheter to remain attached to the implantable, flexible, elongate body when at least 1.5-times a rated pull force is exerted upon the catheter.

In another embodiment, the transfer guidewire assembly also includes a stylet configured to be inserted into the implantable, flexible, elongate body. In another embodiment, the stylet includes an elongate shaft of nickel titanium. In one embodiment, the stylet is integrally formed with the catheter and guidewire. For example, in one embodiment, a portion of the stylet is wrapped around the proximal end of the guidewire. In another embodiment, the wrapped proximal portion of the guidewire is surrounded by an end region of the catheter.

Several embodiments of the invention provide these advantages, along with others that will be further understood and appreciated by reference to the written disclosure, figures, and claims included herein.

### BRIEF DESCRIPTION OF THE DRAWINGS

The operation of the invention will be better understood with the following detailed description of embodiments of the invention, along with the accompanying illustrations, in which:

FIG. 1 shows a diagram of the central systemic veins and how they relate to the cardiac chambers. The left atrium has been catheterized by standard femoral transseptal technique and access to the left subclavian vein has been established using a standard large bore introducer sheath.

FIG. 2 shows one step in one embodiment of placing a catheter from a subclavian vein entry site and having the catheter exit through the same femoral vein access site that was used for the transseptal catheterization.

FIGS. 3A through 3D detail further steps in one embodiment according to the present invention for passing a catheter from a subclavian vein entry site to a femoral vein access site, preparatory to transferring the proximal end of a guidewire from the right femoral vein to a desired access site in the left subclavian vein.

FIGS. 4 through 7 show the steps in one procedure according to the present invention in which a guidewire used for the left atrial catheterization is transferred from the femoral access site to the subclavian access site.

FIG. 8 demonstrates how the guidewire, once transferred, can be stiffened to allow over-the-wire insertion of other devices from the subclavian site

FIG. 9 shows the insertion of a large bore sheath over the transferred wire, through the atrial septum, and into the left atrial site from the subclavian access route.

FIG. 10 demonstrates the placement of an implantable device on the intra-atrial septum from a superior venous approach.

FIGS. 11A through 11D show the insertion of a pacing lead at the right subclavian vein and transfer of the lead to the right femoral vein.

FIGS. 12A through 12C show the transfer of the proximal end of an orally-inserted gastric tube to a nasal insertion site.

FIGS. 13A through 13C detail one embodiment of the invention comprising a guidewire with a movable core mandrel.

FIGS. 14A through 14C detail one embodiment of the invention comprising a guidewire with a proximal movable core mandrel and a fixed distal core.

FIG. 15 is a schematic illustration of the cardiac anatomy with an implanted cardiac rhythm management device with three implanted leads.

FIGS. 16A to 16F depict one embodiment of the invention for transferring an implanted component from one position to another position in the presence of existing implanted components in the cardiovascular system.

FIG. 17 illustrates one embodiment of a sheath with a side port that may be used with the transfer procedure.

FIGS. 18A to 18C depict various views of a coupler device that may be used to join the pull member and lead component during the transfer procedure.

FIGS. 19A to 19C depict the main body of the coupler device of FIGS. 18A to 18C.

FIGS. 20A to 20C depict one locking cap of the coupler device of FIGS. 18A to 18C.

FIGS. 21A and 21B depict another locking cap of the coupler device of FIGS. 18A to 18C.

FIG. 22 illustrates a perspective view of one embodiment of a transfer guidewire.

FIG. 23 illustrates a cross sectional view of a portion of the transfer guidewire of FIG. 22.

FIG. 24 illustrates a perspective view of one embodiment of a sensor lead compatible with the transfer guidewire of FIG. 22.

FIG. 25 illustrates a perspective view of an assembly showing the transfer guidewire of FIG. 22 coupled to the sensor lead of FIG. 24.

FIG. 26 illustrates a cross sectional view of a portion of the assembly of FIG. 25.

FIGS. 27-29 illustrate block diagrams of a flexible elongate body being manipulated within a patient's body to move one end of the elongate body from a first access point to a second access point.

FIG. 30 illustrates a flow chart of a method of manipulating a flexible elongate body within a patient's body.

FIG. 31 illustrates a side view of another embodiment of a transfer guidewire.

FIG. 32 illustrates a proximal end view of the transfer guidewire of FIG. 31.

FIG. 33 illustrates a side view of a stylet compatible for use with the transfer guidewire of FIG. 31.

FIG. 34 illustrates a distal end view of the stylet of FIG. 33.

FIG. 35 illustrates a side view of the transfer guidewire of FIG. 31 coupled to a flexible elongate body.

FIG. 36 illustrates a cross-sectional view of the transfer guidewire, stylet, and flexible elongate body of FIG. 35.

FIG. 37A and FIG. 37B illustrate cross sectional side views of additional embodiments of a transfer guidewire.

FIG. 38 illustrates a side view of a lead transfer slitter in accordance with one embodiment.

FIG. 39 illustrates a side view of the lead transfer slitter of FIG. 38.

### DETAILED DESCRIPTION

Several embodiments of the present invention generally relate to a system and method for performing catheterization of a body structure from a standard catheter insertion site, advancing a guidewire or other flexible member (e.g., an electrical lead, conduit, tube, etc.) into the body structure from that insertion site, and transferring the proximal end of the guidewire to an alternative insertion site while leaving the distal end of the guidewire within the body structure. The transferred guidewire may then be used for the placement of a second device or to perform a desired procedure from the alternative insertion site. Some embodiments relate to methods for standard transseptal puncture of the left atrium from a femoral vein, where the guidewire is then transferred from the femoral insertion site to a subclavian vein insertion site for the implantation of a left atrial pressure-monitoring device. Several embodiments described herein are also generally applicable to other sites of catheter and device insertion. Methods for transferring a medical device or a medical device component, such as a pacemaker lead, between different insertion sites are also provided.

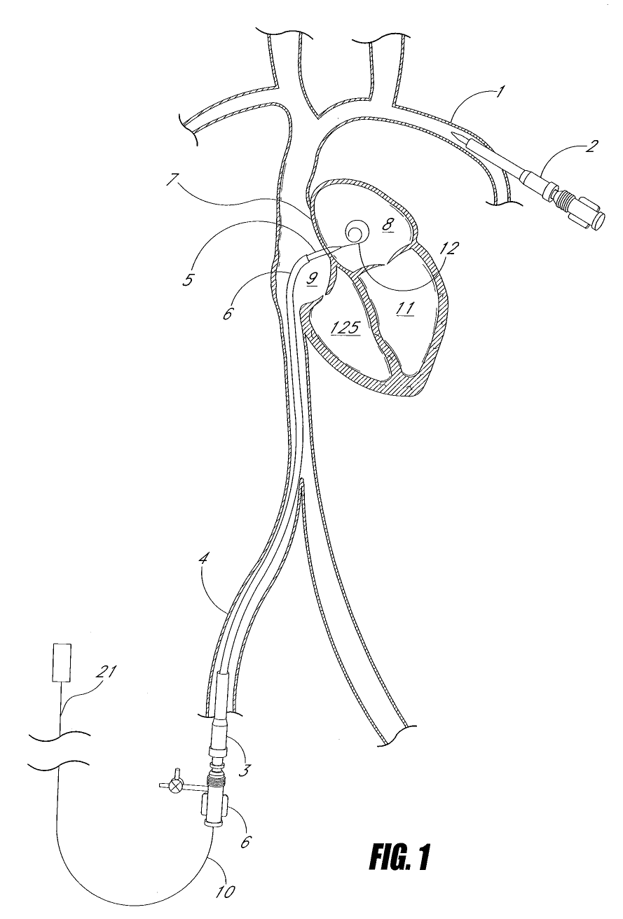

In one embodiment as shown in FIG. 1, the method involves gaining percutaneous or cut-down access into a superior central vein, such as the left subclavian vein 1 as shown, and may involve placing an introducer sheath 2 of appropriate caliber (typically 4-24 French) into the vein 1. A second introducer sheath 3 (typically 4-24 French) is placed in the right femoral vein 4, generally by using either the Seldinger percutaneous method or via surgical cut-down technique, as described by Herbert Chen et al. in “Manual of Common Bedside Surgical Procedures”, 29-76 (Herbert Chen et al. eds., 1996), herein incorporated by reference. From the right femoral access site, a standard transseptal cardiac catheterization is performed using a Brockenbrough needle (not shown), a catheter/dilator 5 and a 6 to 8-French sheath 6, such as a Mullins sheath 6. These initial steps have been described in medical literature, for example, by Charles Davidson et al. in “Heart Disease: A Textbook of Cardiovascular Medicine”, 369-370 (Eugene Braunwald et al. eds., 6th ed. 2001), herein incorporated by reference. The procedure involves performing a needle puncture of the septum 7 using fluoroscopic or ultrasonic visualization of the atrial septal anatomy. Once the puncture of the intra-atrial septum has been performed, the catheter/dilator 5 is advanced over the needle and into the left atrium 8. Ultimately, the Mullins sheath 6 can be advanced over the dilator into the left atrium 8, and the needle and dilator can be entirely removed from the sheath. If communication between the left atrium 8 and the right atrium 9 already exists, such as the presence of patent foramen ovale (PFO) or an atrial septal defect (ASD), access to the left atrium 8 can be performed without transseptal needle puncture and merely by catheter and guidewire manipulation.

In one embodiment, after successful cannulation of the left atrium 8 from the femoral route, a guidewire 10 with a length between about 150 cm to about 300 cm can be placed in the left atrium 8 through the Mullins sheath 6. In another embodiment, the guidewire 10 has a length between about 180 cm to about 280 cm. In another embodiment, the guidewire 10 has a length between about 200 cm to about 260 cm. In yet another embodiment, the guidewire 10 has a preferred length of between about 220 cm to about 250 cm, preferably about 240 cm. The guidewire 10 may also have a length of less than about 150 cm or greater than about 300 cm. In one embodiment, the guidewire 10 includes a moveable or removable core mandrel. Such guidewires include, but are not limited to, a stiffer type of movable core guidewire with a tapered tip on the distal core. In one embodiment, the guidewire distal portion 12 is soft and curled, and can be coiled in either the left atrium 8, left ventricle 11, left atrial appendage (not shown), or a pulmonary vein (not shown) to provide a stable distal position. One skilled in the art will understand that many types of such coils can be used to achieve a stable anchoring position for the distal end of the guidewire 10. In one embodiment, the core can be at least partially pulled back to increase the coiling propensity of the wire. The Mullins sheath 6 or catheter is then withdrawn while maintaining the distal guidewire 12 position.

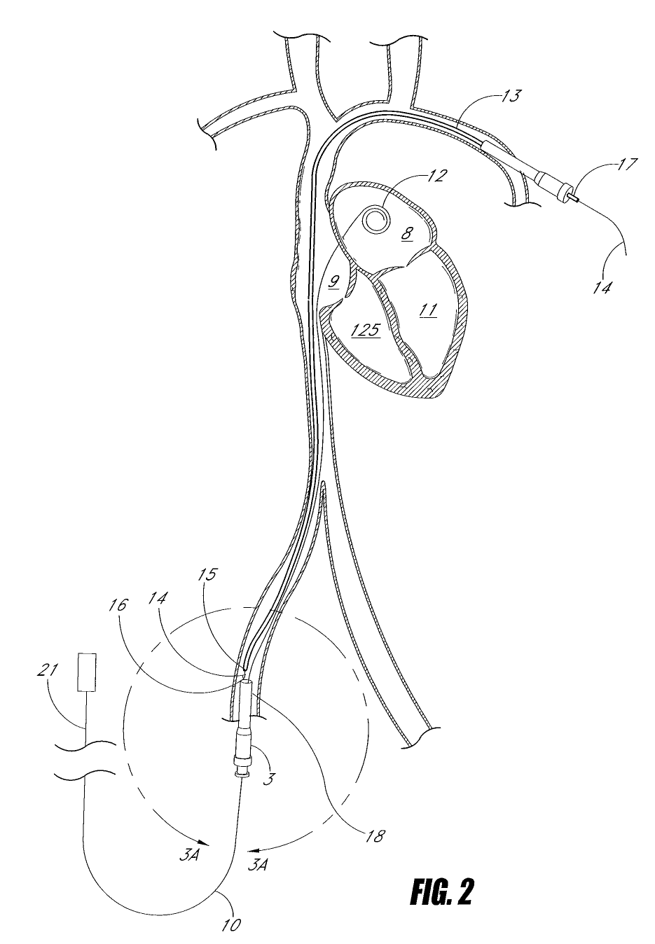

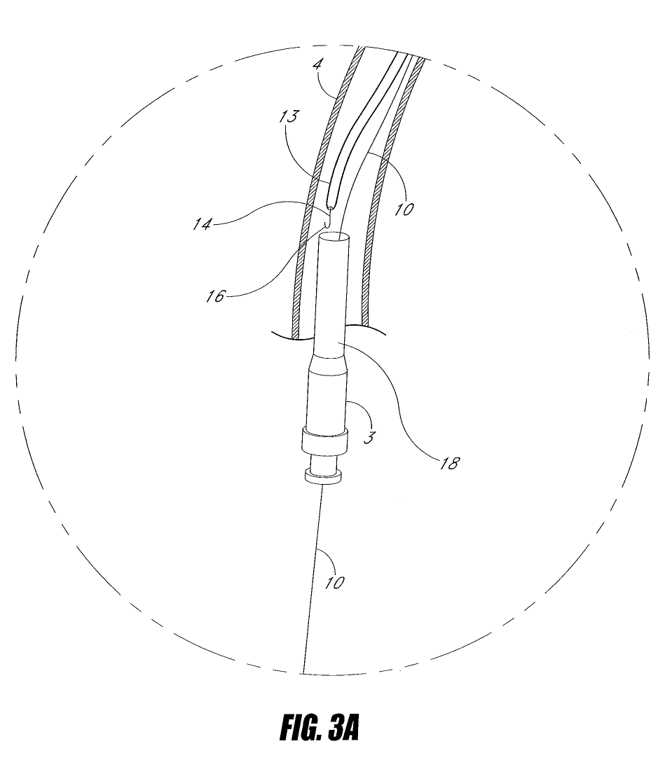

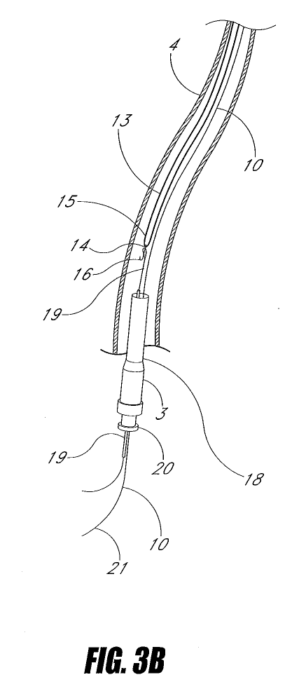

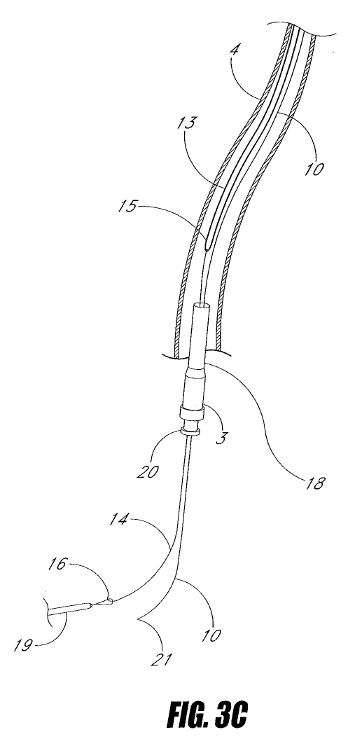

As shown in FIG. 2, in one embodiment, a torqueable catheter 13 is inserted through the subclavian vein sheath 2 over a standard guidewire 14 (diameter typically 0.025-0.038 inches). In one embodiment, the catheter 13 has a diameter of about 4 French to about 6 French and length of about 80 cm to about 100 cm. In one embodiment the catheter 13 has a tip 15 configured with a bend near the distal end, such as a “multi-purpose”, “Judkin's right”, Right Coronary Bypass or “Cobra” shape catheter that allows the tip to be steered by rotating the catheter. Skilled artisans will understand that catheters with a variety of distal tip shapes may be used to enhance steerability through branching or tortuous anatomy. Referring now to a close-up of the femoral access area shown in FIG. 3A, the wire tip 16 may be straight, or it may have a “J”, angled or a bendable distal tip that can be used for steering. One skilled in the art will understand that several shapes and curvatures for the wire tip may be used in accordance with several embodiments of the present invention. The wire 14 and catheter 13 are advanced and manipulated by applying a torque force to the proximal shaft 17 of the catheter 13, wire 14, or both, until they engage the distal end 18 of the femoral vein sheath 3. Care should be taken to minimize entangling the catheter 13 around the previously placed guidewire 10 extending from the left atrial site 8 through the femoral sheath 3.

As shown in FIG. 3B, if difficulty is encountered entering the distal sheath 18 of the femoral vein 4 with the tip 16 of the superiorly placed guidewire 14, the guidewire tip 16 can be grabbed with a commonly available “goose neck” snare 19 (e.g., such as snares available from Microvena Corp., MN) inserted into the femoral sheath 3 and then pulled through the sheath 3 until the distal tip 16 of the guidewire 14 exits through a hemostasis valve 20 of the femoral sheath 3 at the patient's groin, as depicted in FIG. 3C. It may also be helpful to use a thin walled introducer (not shown) placed over the inferiorly inserted guidewire through the hemostasis valve 20 to facilitate the passage of the superiorly placed guidewire 14 and catheter 13 through the hemostasis valve 20. In one embodiment, once the distal tip 15 of the superior catheter 13 exits the femoral vein sheath 3, as depicted in FIG. 3D, the superiorly placed guidewire 14 is removed from the superior catheter 13.

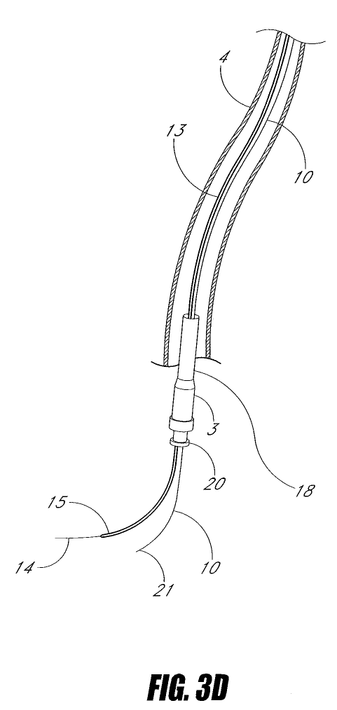

In one embodiment, as shown in FIG. 4, the inferiorly placed guidewire 10, whose distal portion 12 is located in the left atrium 8, is configured so that the proximal end 21 of this guidewire 10, after removing its movable core, may be inserted into the distal tip 15 of the superior catheter 13 exiting the femoral sheath 3. In one embodiment, removal of the movable core advantageously increases the flexibility of the wire body so it will not be plastically deformed (e.g., kinked) during subsequent manipulations. In another embodiment, a small kink may be tolerated. In yet another embodiment, a single-piece guidewire constructed from superelastic nickel titanium (e.g., nitinol) or other material with similar properties as known in the art may be used to provide a guidewire 10 that is more kink-resistant than traditional stainless steel guidewires and does not utilize a moveable core mandrel. One skilled in the art will understand that many such guidewire configurations exist and may be applicable. The proximal end 21 of this guidewire 10 is advanced into the superior catheter 13 until its proximal end 21 exits from the proximal end 17 of the subclavian catheter 13. Thus, the proximal end 21 of the transseptal wire 10 is “backloaded” into the distal tip 15 of the catheter 13 exiting the femoral vein sheath 3 and is advanced until it protrudes from the proximal shaft 17 of the catheter 13.

In another embodiment, the catheter tip 15 is advanced to the inferior insertion site in the right femoral vein 4 but it does not exit the inferior introducer sheath 3. The guidewire 10 may be backloaded into the distal tip 15 of the catheter 13 under fluoroscopic or ultrasonic guidance, or by using a snare 19 inserted through the catheter 13 from its superior proximal end 2.

In yet another embodiment, the proximal end 21 of the inferior guidewire is docked into or attached to the distal end of the superior guidewire 14 such that the two wires 10, 14 form a single continuous loop from the superior subclavian entry site, out through the femoral sheath 3, back through the femoral sheath, and ending in the target site 8. Other docking mechanisms may be used to attach the two guidewires 10, 14 together.

In one embodiment, advancement of the guidewire 10 through the catheter 13 is continued until a small loop 22 is left exiting the femoral sheath 3, as depicted in FIG. 4. Referring to FIG. 5, the catheter 13 and guidewire 10 are withdrawn from the superior insertion site in the left subclavian vein 1. In one embodiment, the catheter and guidewire are withdrawn as a unit. In another embodiment, the catheter and guidewire may be manipulated individually during withdrawal to alter their relative positions as indicated to the operator by visual, auditory, mechanical, or other means, such as by fluoroscopy or ultrasonography. A thin-walled introducer (not shown) may be advanced over this loop into the hemostatic valve 20 of sheath 3 to facilitate pulling the loop 22 through the valve 20 and into the catheter 13. In one embodiment, the movable core is withdrawn into the catheter 13 so that the wire exiting the catheter 13 and sheath 3 in the groin contains no core and has increased flexibility during the transfer maneuver just described.

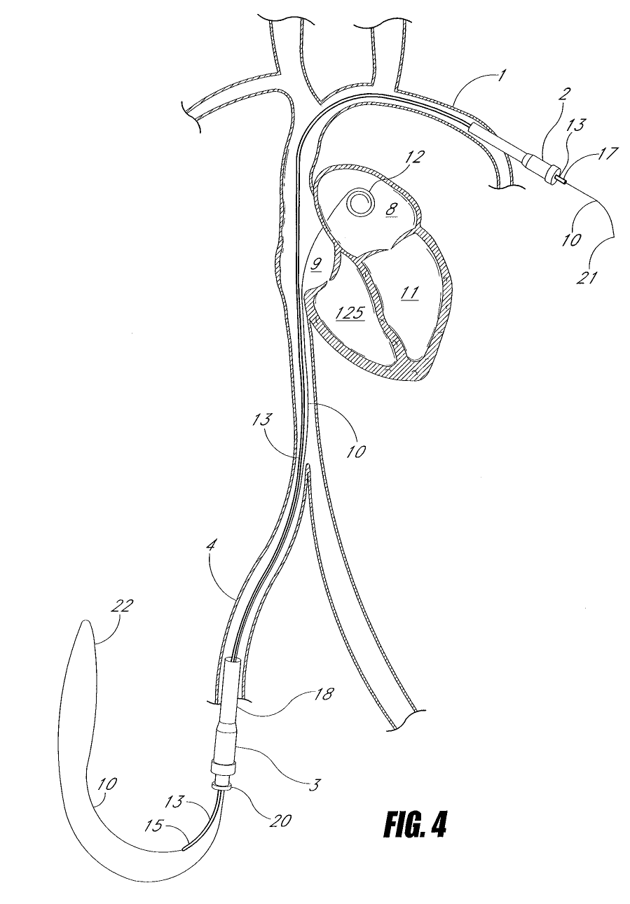

In one embodiment, the guidewire 10 is sufficiently flexible without the core such that it is capable of creating at least a tight 180 degree bend 23 in the venous system without injuring the wire or the venous system, as illustrated in FIG. 6. In another embodiment, the guidewire 10 is capable of bending at least about 180 degrees in a vascular lumen between about 0.5 cm to about 4 cm in diameter, preferably between about 0.75 cm to about 1.5 cm in diameter, and more preferably about 1 cm in diameter.

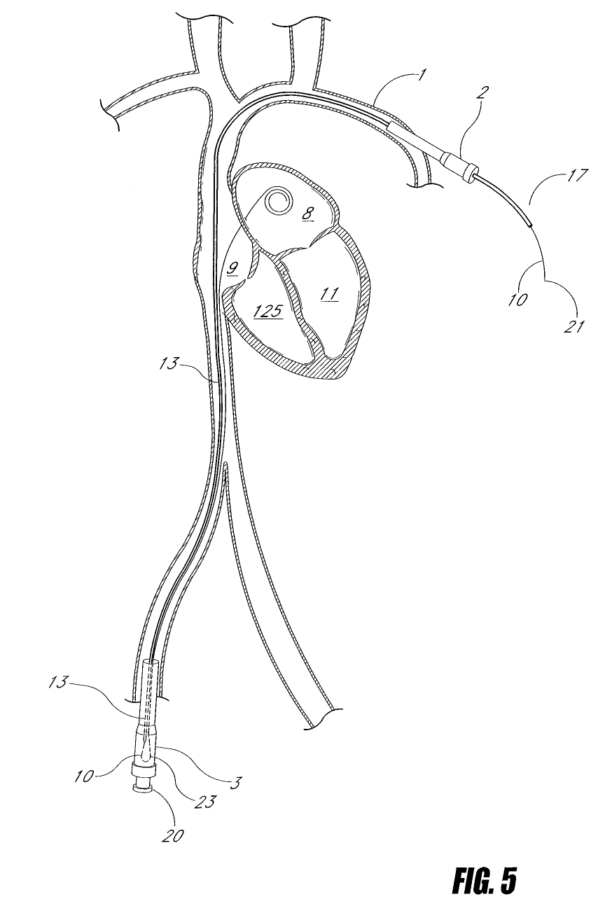

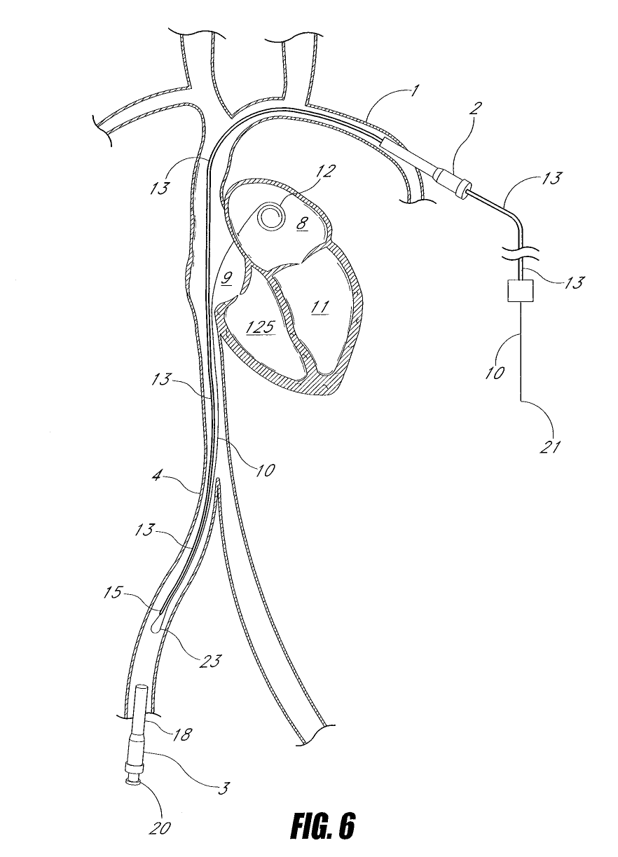

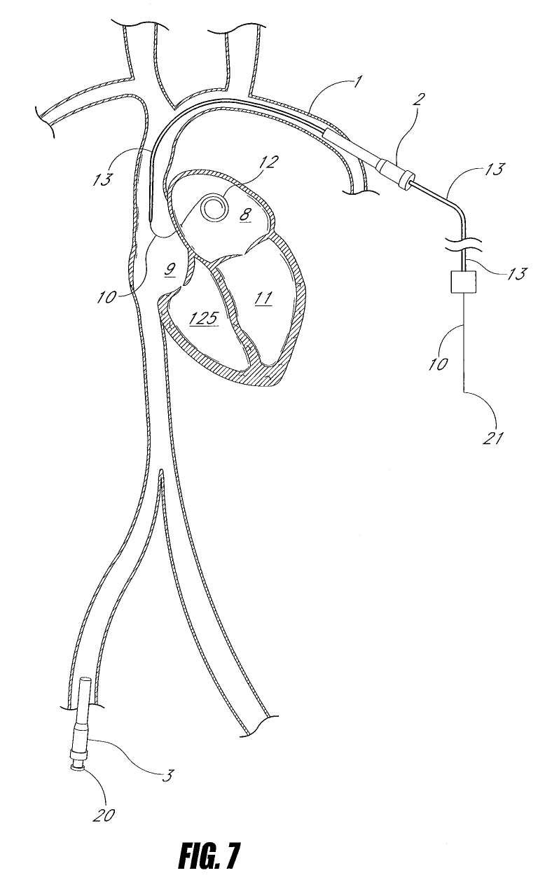

As shown in FIG. 7, in one embodiment, as the catheter 13 is removed, the distal position of the guidewire 10 is maintained in the left atrium 8. Once the catheter 13 is removed, only the guidewire 10 exits the sheath 2 in the subclavian vein 1. The wire 10 may have a minimal kink where it had previously formed a tight loop 23, but this area of the kink is external to the patient, having exited the subclavian sheath 1. The movable inner core mandrel is re-advanced such that it crosses the intra-atrial septum 7 and is in the left atrium 8 to help facilitate catheter transfer over this stiffened guidewire 10, as shown in FIG. 8.

Referring now to FIG. 9, the subclavian sheath 2 (not shown) can be replaced with a large bore introducer 24, which is advanced over the guidewire 10 and into the left atrium 8. In one embodiment, the large bore introducer 24 is of the “peel away” type, commonly used by skilled artisans for placement of implantable medical devices with a larger proximal diameter, such as an implantable pacing or defibrillator lead that is connectable to a proximal housing 25 (FIG. 10), such as a pacemaker or defibrillator generator. In one embodiment, the introducer 24 may facilitate placement of one or more medical devices 25 and/or devices for closure of the left atrial appendage. Medical devices include, but are not limited to, a pacemaker lead, a patent foramen ovale closure device, and a device for measuring left atrial pressure 26, shown in FIG. 10. In another embodiment, the guidewire, if positioned into the left ventricle, may be used to advance a mitral valvuloplasty balloon. One skilled in the art will understand that several diagnostic and therapeutic applications can be used in accordance with several embodiments of the present invention.

In a further embodiment of the invention, the inferior guidewire 10 is not positioned in any particular target site when the guidewire transfer is performed, but is advanced to the target site after the guidewire transfer is performed. In another embodiment, the distal position of the guidewire 10 is not maintained in any particular position or body structure but a middle portion of the guidewire 10 passes through and is constrained by a body structure, such as the intra-atrial septum. This body structure may act as a pivot point to allow movement of the guidewire portion between the pivot point and the proximal end of the guidewire 10 while constraining at least a portion of the movement of the guidewire 10 at the body structure.

Several embodiments of the present invention are particularly advantageous because of their applicability to the general case of transferring a wire from one insertion site in the venous or arterial circulation to another exit site for that wire in the same circulation. Other insertion sites that may be used with several embodiments of the invention include, but are not limited to, the radial arteries, dorsalis pedis arteries, axillary arteries and internal jugular veins. Access to these sites are known to those in the art and are described by Herbert Chen et al. in “Manual of Common Bedside Surgical Procedures”, 29-76 (Herbert Chen et al. eds., 1996), herein incorporated by reference. Several embodiments of the invention also provide for other target sites, including the right ventricle, left ventricle, pulmonary arteries, pulmonary veins, renal arteries, renal veins, portal veins, hepatic arteries, carotid arteries, jugular veins, axillary arteries, axillary veins and pathological sites such as an abdominal aortic aneurysm.

Several embodiments of the invention are also advantageous because of their general applicability to the concept of transferring the proximal end of a guidewire from a first insertion site to a second insertion site, after inserting the distal end of the guidewire from the first insertion site toward a target site or in proximity of a target site. In one embodiment, the insertion and transfer of a guidewire defines a series of pathways in the body taken by the proximal and distal ends of the guidewire. The initial insertion of the distal end of the guidewire is capable of defining a first pathway between the first insertion site and a target site. The transfer of the proximal end of the guidewire from the first insertion site and the second insertion site is capable of defining a second pathway taken by the proximal end of the guidewire. By transferring the proximal end of the guidewire, a third pathway is then defined along the new guidewire position, from the second insertion site to the target site. The third pathway may be used to access the target site.

In some embodiments of the invention, a conduit is placed between the first insertion site and second insertion site to facilitate transfer of the proximal end of the guidewire. In the preferred embodiment, the conduit includes a catheter inserted from the second insertion site to first insertion site, but one skilled in the art will understand that the conduit may comprise any structure that provides a lumen generally between the first insertion site and the second insertion site and that the conduit may be inserted between the insertion sites in other ways. For example, the conduit may be placed from the first insertion site to the second insertion site. In other embodiments, a conduit is not used to transfer the proximal end of the guidewire and the guidewire is transferred by other devices, such as a snare that pulls the proximal end of the guidewire from the first insertion site to the second insertion site.

In some embodiments, portions of the first pathway and the third pathway may overlap. For example, in one embodiment, the first insertion site is the right femoral vein, the second insertion site is the right subclavian vein and the target site is the left atrium. The first pathway from the right femoral vein to the left atrium, and the third pathway, from the right subclavian vein to the left atrium, share a common distal portion from the intra-atrial septum to the left atrium. The most proximal point common to both the first and third pathways define a pivot point whereby the distal portions of the first and third pathways are constrained to at least partially overlap and where the portions proximal to the pivot point do not overlap. In one embodiment, the second pathway taken by the proximal end of the guidewire does not cross or intersect the pivot point or the target site, but may pass through structures that the first and third pathways also pass through. Such structures are defined as junction areas and typically, but not always are situated proximal to the pivot point and/or target area. In the example mentioned above, all three pathways will pass through a junction that includes the right atrium.

In another embodiment, a patient is treated by introducing a guidewire into a patient at a first access site and advancing the guidewire translumenally to a target site. The flexibility of at least a portion of the guidewire is adjusted and is transferred to a second access site. In one embodiment, the adjustment of the guidewire flexibility is performed by moving a core wire within the guidewire. In another embodiment, the flexibility is adjusted by advancing a tubular support around the outside of the guidewire.

In another embodiment, a method for accessing a target site is provided, where a guidewire is introduced into a patient through an introduction site, the guidewire having a first, reduced flexibility. The guidewire is then adjusted to a second flexibility to advantageously externalize at least a portion of the guidewire through a different introduction site of the body. A catheter is then introduced along the guidewire.

In one embodiment, this procedure may be used to cannulate the coronary sinus in the right atrium from the usual superior venous approach. Using the methodology of one embodiment of the present invention, once a guidewire is placed in the coronary sinus, a catheter can be threaded from an inferior venous approach to exit from the superior introducer site. A withdrawal of the guidewire core creates a soft bend, followed by backloading of the wire into the distal end of the catheter until it exits the proximal end of the catheter shaft in the groin. The catheter is subsequently withdrawn and accomplishes transfer of the wire from a superior insertion site to an inferior insertion site. This approach could be used for placing the left ventricular lead of a cardiac resynchronization pacemaker (biventricular pacemaker) when the rhythm management system generator must be placed in the lower abdominal wall. Similar approaches can be performed on the arterial side of the circulation as well. In accordance with many embodiments of the current invention, similar approaches can be performed when cannulating any orifice in any hollow viscus in the body of an organism, including but not limited to the gastrointestinal system, urinary system, reproductive system and central nervous system. For example, in some embodiments of the invention, the oropharynx, nasopharynx, rectum, urethra may be used as insertion sites. In other embodiments of the invention, artificial locations, such as a ventriculoperitoneal shunt, nephrostomy tube or gastric tube, may be used as insertion sites.

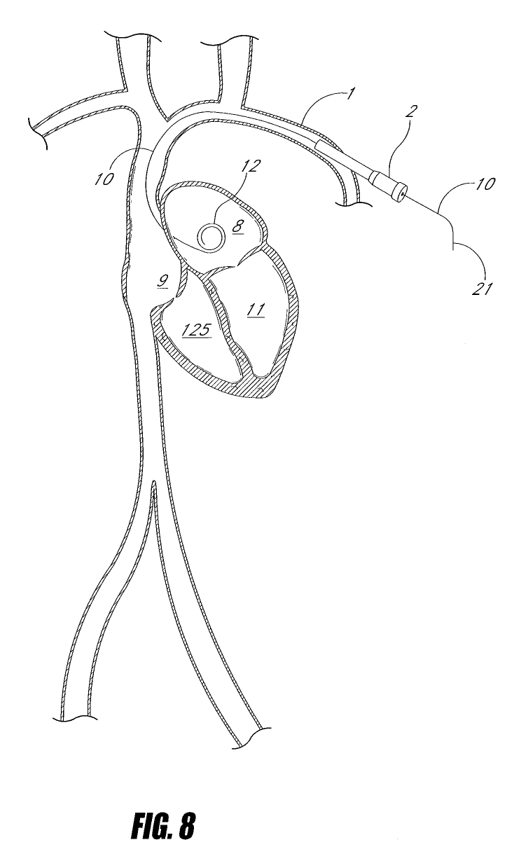

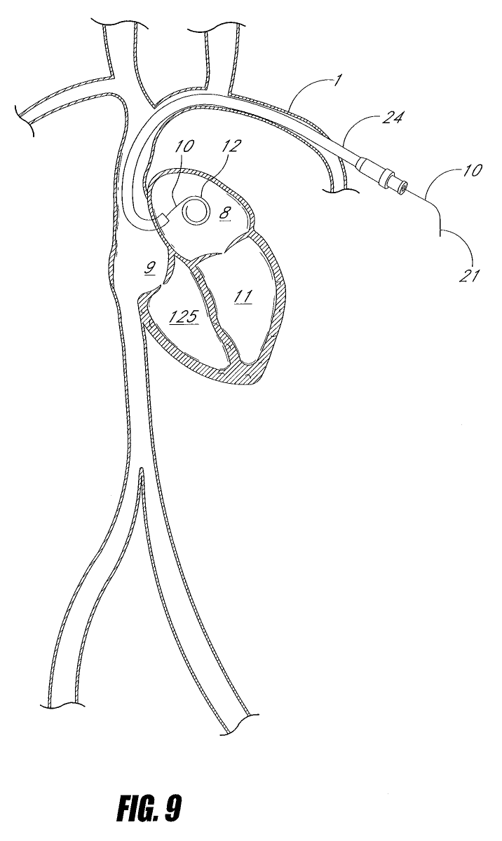

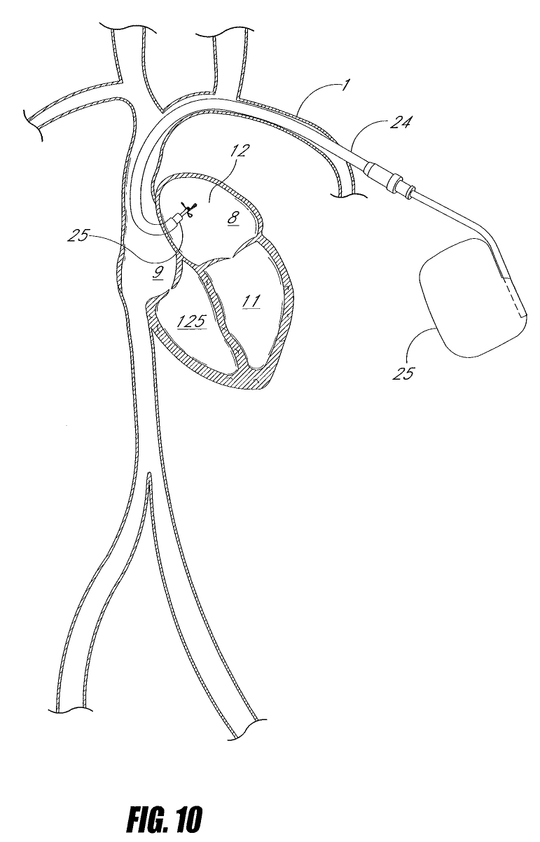

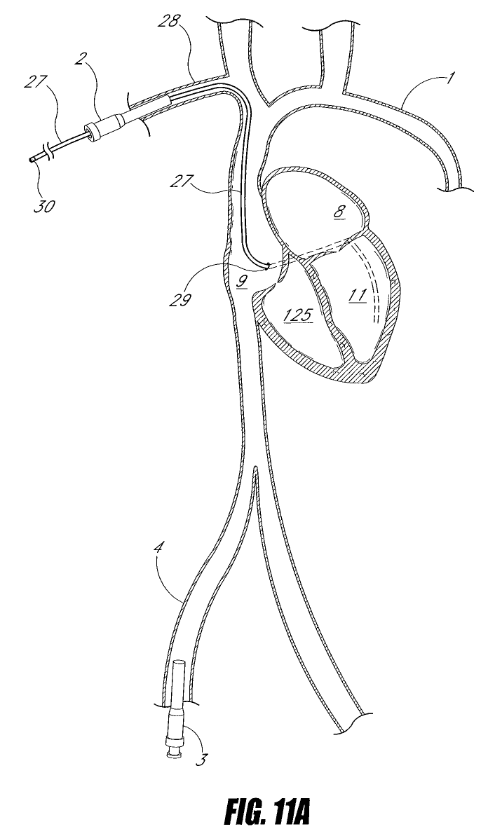

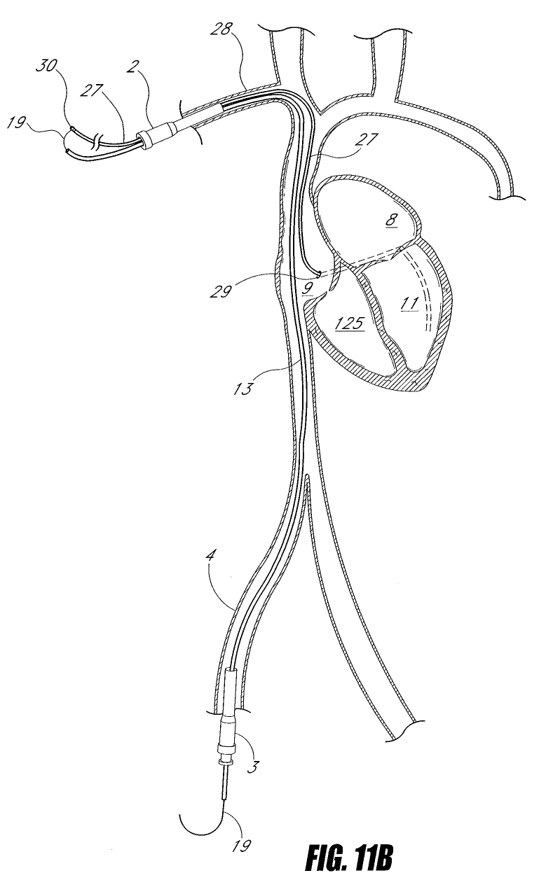

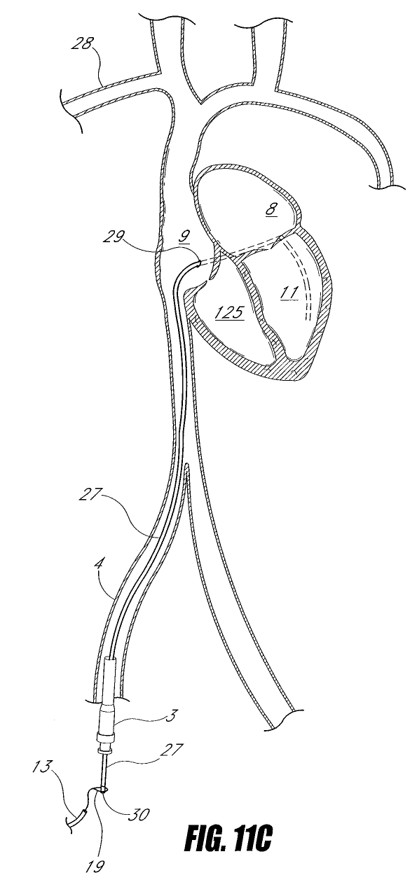

In addition to embodiments of the invention for transferring guidewires, several embodiments of the invention may be adapted to provide for the transfer of at least a portion of a device from one insertion site to another insertion site, with or without the device on a guidewire. Devices capable of such transfer include but are not limited to sensor leads, pacing leads, catheters and any other medical device or portion of a medical device that is capable of movement through a body lumen of an organism. For example, FIG. 11A depicts the insertion of a left ventricular lead 27 of a biventricular pacemaker described previously. In one embodiment, the lead 27 is inserted through a first introducer or sheath 2 at a first insertion site (at the right subclavian vein 28) and into the coronary sinus 29 in the right atrium 9. A catheter 13 is inserted into a second introducer or sheath 3 at a second insertion site (at the right femoral vein 4), through the inferior vena cava, right atrium and superior vena cava and externalized through the first insertion site (see FIG. 11B). For example, in one embodiment, the catheter 13 is advanced over a guidewire, which has previously been inserted into the patient such that the guidewire extends between the first and second insertion sites. The lead 27 is backloaded into the catheter 3. For example, in one embodiment, the proximal end of the lead 27 is slid into the end of the catheter 13 that exits the patient at the first insertion site. In other embodiments, the proximal end of the lead 27 is attached to the catheter 13 end in a manner that permits the lead 27 to be withdrawn through the patient's body to the second insertion site. Once attached, the catheter 13 and lead 27 are withdrawn from the patient's body via the second insertion site.

In addition, if the lead 27 lacks sufficient length to be backloaded into the catheter 13 or if the lead connector 30 cannot fit through the catheter 13 lumen, a snare 19 (such as a guidewire with a snare at one end, or other device capable of releasably engaging the proximal end of the lead 27) may be extended between the first and second insertion sites and used to pull the proximal end of lead 27 from the first insertion site to the second insertion site. FIG. 11B shows a snare guidewire 19 extending through a catheter 13 and attached to the proximal end of the left ventricular lead 27. FIG. 11C shows the snare 19, catheter 13, and the lead 27 withdrawn from the right femoral insertion site. The lead 27 is released from the snare 19 and connected to the biventricular pacemaker 31, as demonstrated in FIG. 11D.

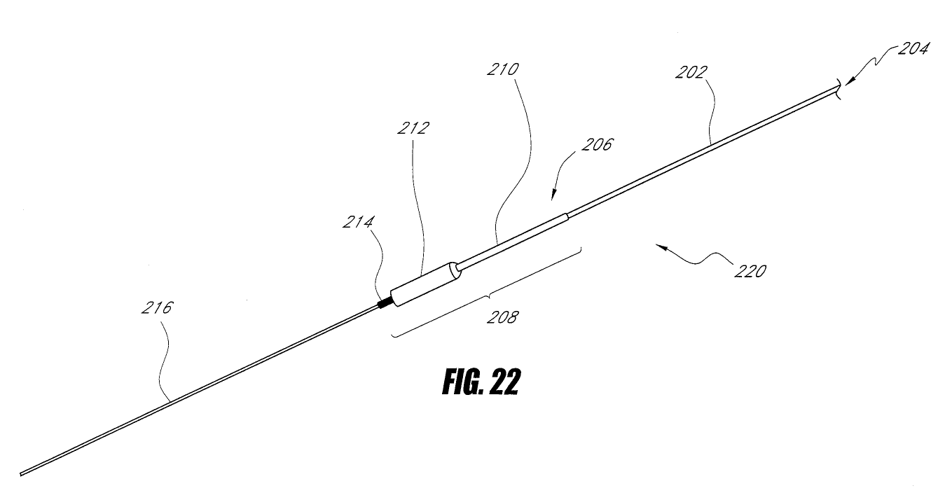

In another embodiment, an extension device such as a guidewire or stylet is removably engaged to the proximal end of the lead 27 to allow the distal end of the lead to be advanced to its target location even when the length of the lead is shorter than the distance from the first insertion site to the target location. FIGS. 22-26 and the related discussion below describe embodiments that can include such a stylet. The proximal end of the extension device may then be transferred to a second insertion site closer to the target site than the length of the lead, and the extension device may then be withdrawn so that the proximal end of the lead is externalized at the second insertion site. One example of this embodiment is the transfer of a short 45 cm left atrial pacing and/or pressure sensor lead inserted through a first insertion site in the femoral vein for transfer to a second insertion site in a subclavian vein. The first insertion site is more than 45 cm from the left atrium and will cause the proximal end of a lead to enter the body when the distal end of the lead is positioned at the target site. It will be clear to one skilled in the art that accessing the left atrium, via the atrial septum, may be easier and safer from the first insertion site, but that the ultimate desired location for the proximal end of the lead may be the subclavicular region. Furthermore, the skilled artisan will appreciate that it is undesirable to use a lead with sufficient length to span the entire distance from the femoral vein to the left atrium because once the lead is transferred to the second, closer, insertion site the excess length would have to be coiled and implanted within the patient.

In some embodiments, the procedure for transferring one or more guidewires, sensors leads, or other medical devices may be performed in patients with pre-existing components within the vasculature or body lumen. The pre-existing implant components may have been implanted either in a prior procedure or earlier in the same procedure. One example of this is a patient with a pre-existing implanted cardiac pacemaker who is undergoing the implantation of a left atrial pressure sensor. In such circumstances, performing the guidewire transfer or lead-transfer procedure may cause snagging, dislodgement or damage to the existing leads or sensors, or to the components being implanted. Such risks may be reduced by providing a protected pathway for the guidewires and/or leads involved in the transfer procedure. The protected pathway provides a space for the transfer procedure to take place while excluding at least a portion of the existing vascular or implanted components from interfering with the transfer procedure. The common protected pathway, which may be in the form of a protective sheath or other conduit, can provide a barrier between the transfer procedure and the existing implanted leads or sensors. The protective sheath may be placed along at least a portion of the transfer pathway to reduce interference between the existing components and the new components. The protected pathway need not extend along the entire length between the two entry sites or extend along the entire section of the body lumen or body cavity where the existing components reside.

In one embodiment, a protective sheath may be placed from a left subclavian access site to the superior or inferior vena cava, or more distally to the right ventricular apex, the left ventricle coronary sinus and/or the right atrial appendage, for example. Other target and insertion sites, as mentioned elsewhere, may be used. The selection of the insertion sites for the protective sheath may be based upon a variety of factors, including avoidance of stenotic lesions, valvular insufficiency and unstable plaques. A sleeve or other covering may be used instead of or in addition to the protective sheath.

In other embodiments, due to the size of the new components to be implanted, providing a protective sheath or barrier between the new components and existing components may be impractical. In still other instances, impracticality may result from the proximity between the target implantation site and the existing leads or sensors. In these circumstances, the risks of snagging or dislodging of the existing leads or sensors may still be reduced by providing a protected common pathway at least during the insertion of the components involved in the transfer procedure. Even though the protected common pathway is removed prior to the pullthrough step of the transfer procedure, for example, as long as the guidewires or other components involved in the transfer procedure are protected during insertion from looping or intertwining with the existing sensor leads by a protective sheath, the transfer pathways formed during insertion will still be free of looping or intertwining even after the protective sheath is removed.

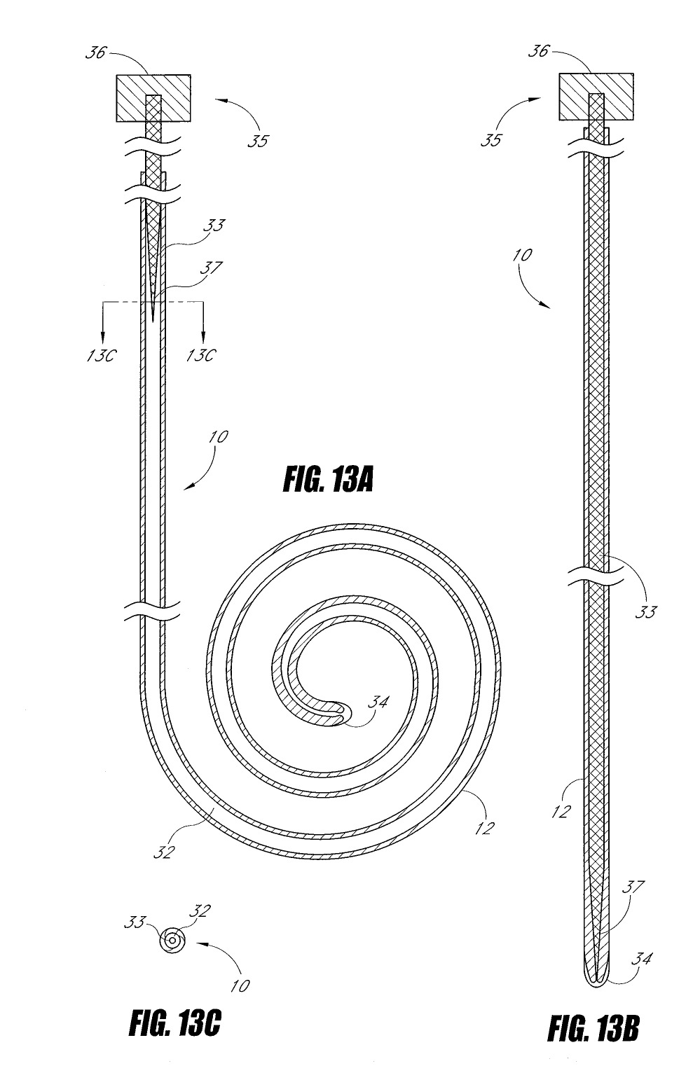

Referring to FIG. 15, in one embodiment, the transfer procedure is performed in a patient with a pre-existing cardiac rhythm management (CRM) implant 100 with leads 102, 104, 106 suitable for delivering multi-chamber stimulation and shock therapy. Although the leads discussed below have been implanted at traditional implantation sites for CRM leads, one of skill in the art will understand that in other embodiments, the guidewire or lead transfer procedure may involve anatomical locations other than those discussed below. The transfer procedure may also be used with leads implanted at other cardiac sites, non-cardiac sites or non-vascular sites.

Referring still to FIG. 15, in one embodiment, the CRM device 100 is coupled to an implantable right atrial lead 102 that is implanted in the right atrial appendage for sensing atrial cardiac signals and to provide right atrial chamber stimulation therapy. To sense left atrial and ventricular cardiac signals and to provide left-chamber pacing therapy, the CRM device 100 is coupled to a “coronary sinus’ lead 104 designed for placement in the coronary sinus region 108 via the coronary sinus opening 110 for positioning a distal electrode adjacent to the left ventricle 11 and/or additional electrodes adjacent to the left atrium 8. As used herein, the phrase “coronary sinus region” refers to the vasculature of the left ventricle 11, including any portion of the coronary sinus 108, great cardiac vein, left marginal vein, left-posterior ventricular vein, middle cardiac vein, and/or small cardiac vein or any other cardiac vein accessible by the coronary sinus. The coronary sinus lead 104 according to one embodiment is configured to receive atrial and ventricular cardiac signals and to deliver left ventricular pacing therapy using at least a left ventricular tip electrode 112, left atrial pacing therapy using at least atrial ring electrode 114 and shocking therapy using at least a left atrial coil electrode 116. The CRM device 100 is also depicted with an implanted right ventricular lead 106 located in the right ventricular apex 118. The right ventricular lead 106 typically includes a right ventricular tip electrode 120 at the apex 118, along with a right ventricular ring electrode 122 and coil electrode 124 positioned in the right ventricle 125 and a superior vena cava coil electrode 124 positioned in the superior vena cava 132. The right ventricular lead 106 is capable of receiving cardiac signals and delivering stimulation to the right ventricle 125. The housing 126 of the CRM device 100 is typically implanted in the subcutaneous tissue of the upper torso, and is often referred to as the “can”, “case” or “case electrode”. The CRM device housing 126 may be programmably selected to act as the return electrode for all “unipolar” modes. The housing 126 may further be used as a return electrode alone or in combination with one or more of the coil electrodes for shocking purposes. In other embodiments, the leads may have other target sites, including other locations within the cardiovascular system, including epicardial or pericardial leads, as well as non-cardiovascular locations such as the chest wall or pleural cavity, where a physiological sensor may be used to assess physical activity level for activity-responsive pacing. The lead location will vary depending upon the location of the device housing(s) as well as the desired implant locations of the lead locations. The leads need not originate from a housing implanted in the left upper chest regions. Other implantation locations or origination sites may be used with the transfer procedure.

In other embodiments, the pre-existing implanted components may involve vascular devices unrelated to cardiac rhythm management, and/or may lack a housing and/or lack lead-like members. One such example is a percutaneously implantable annuloplasty ring as that described in U.S. Patent Pub. No. 2007/0051377, herein incorporated by reference in its entirety.

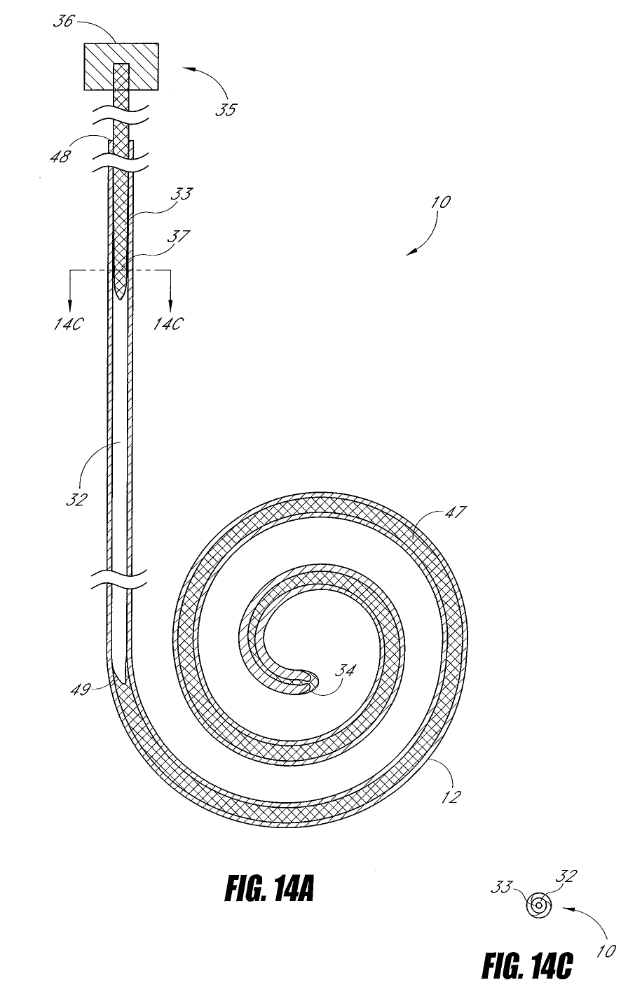

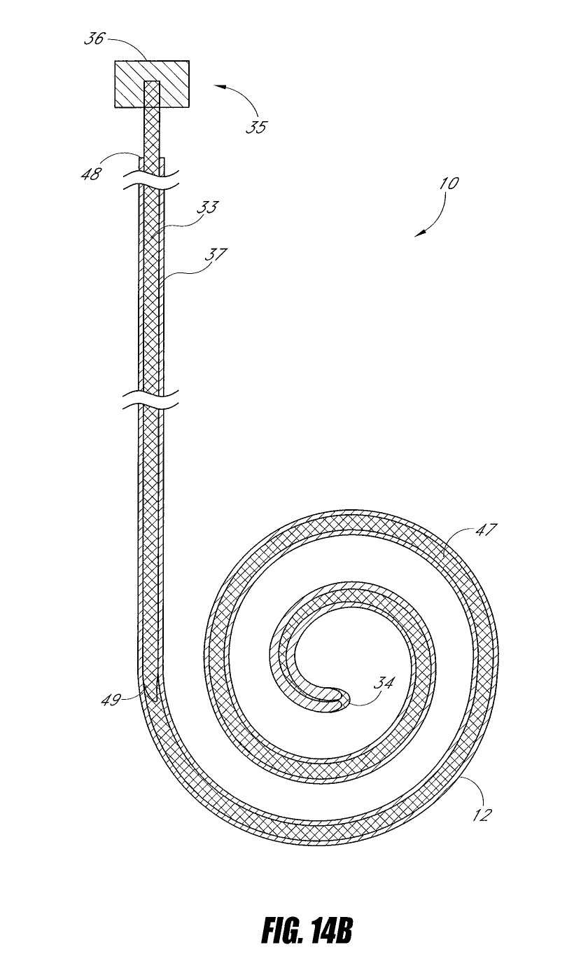

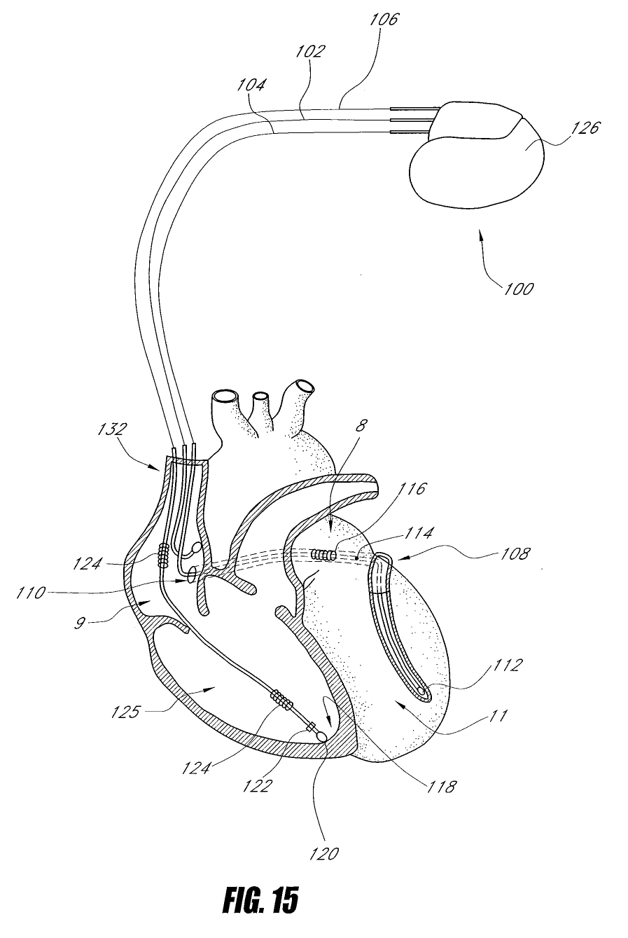

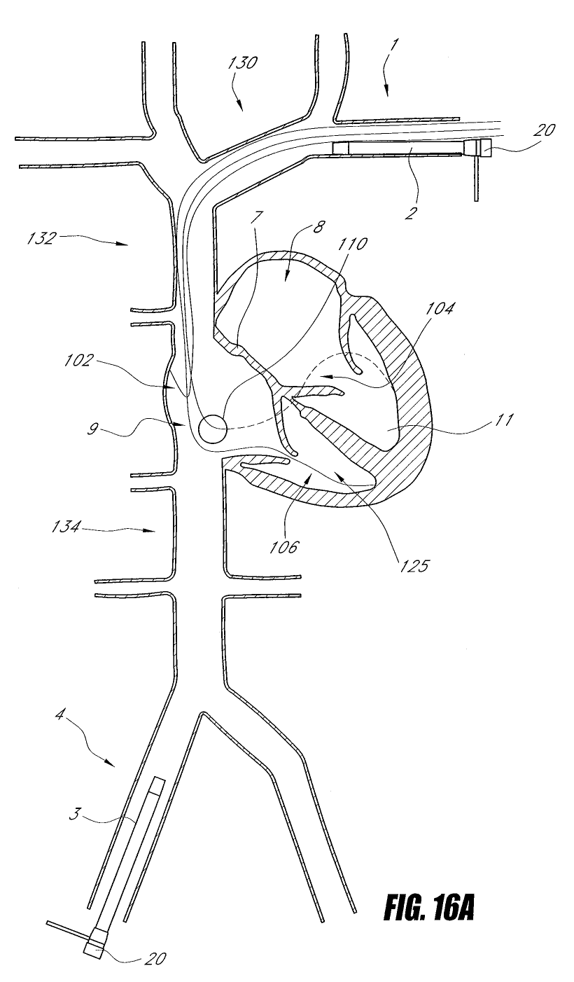

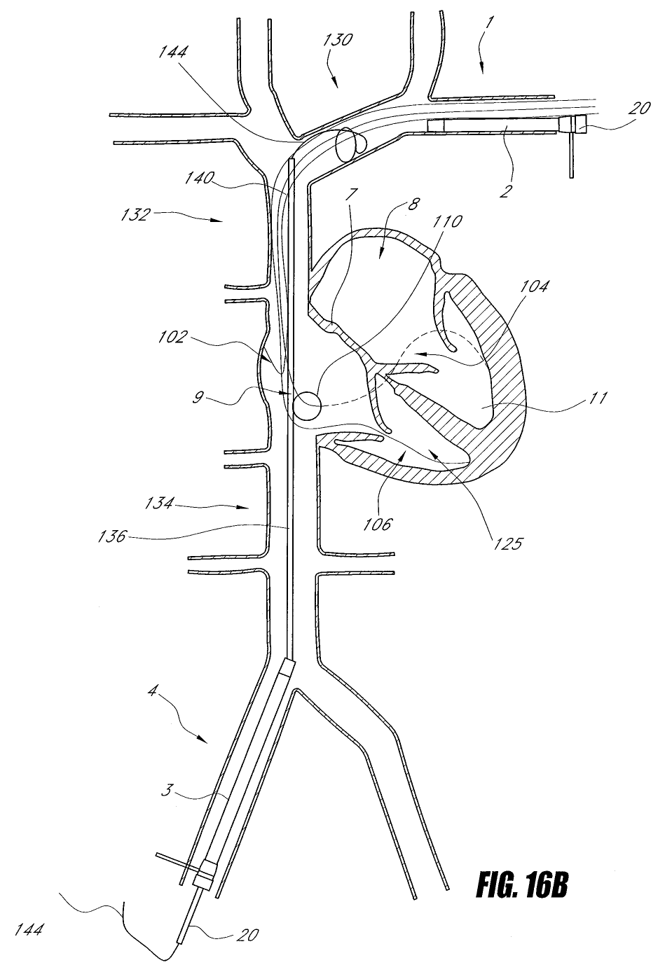

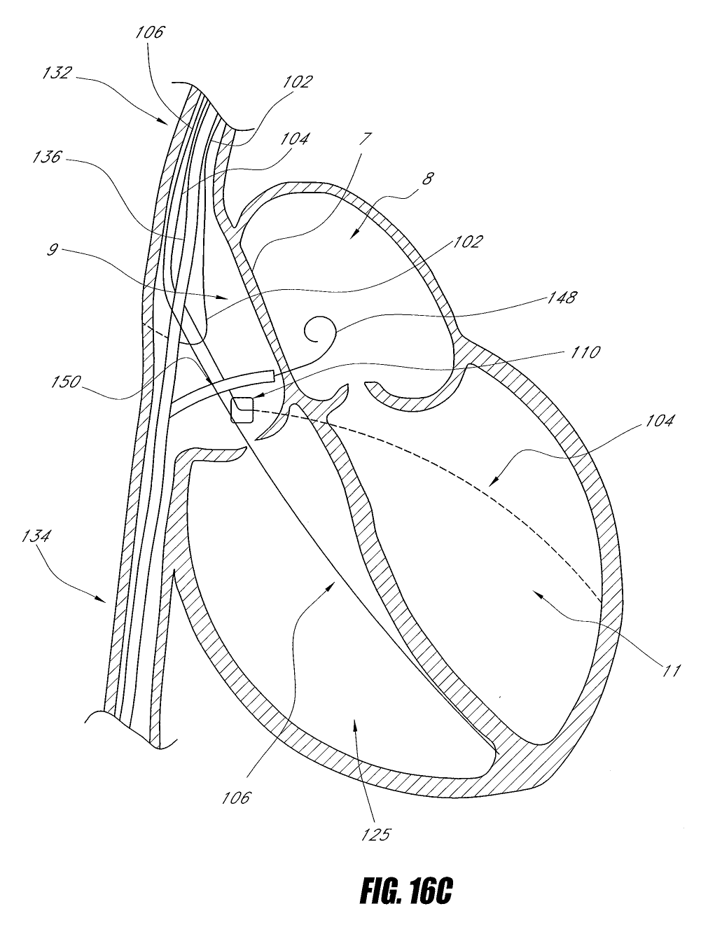

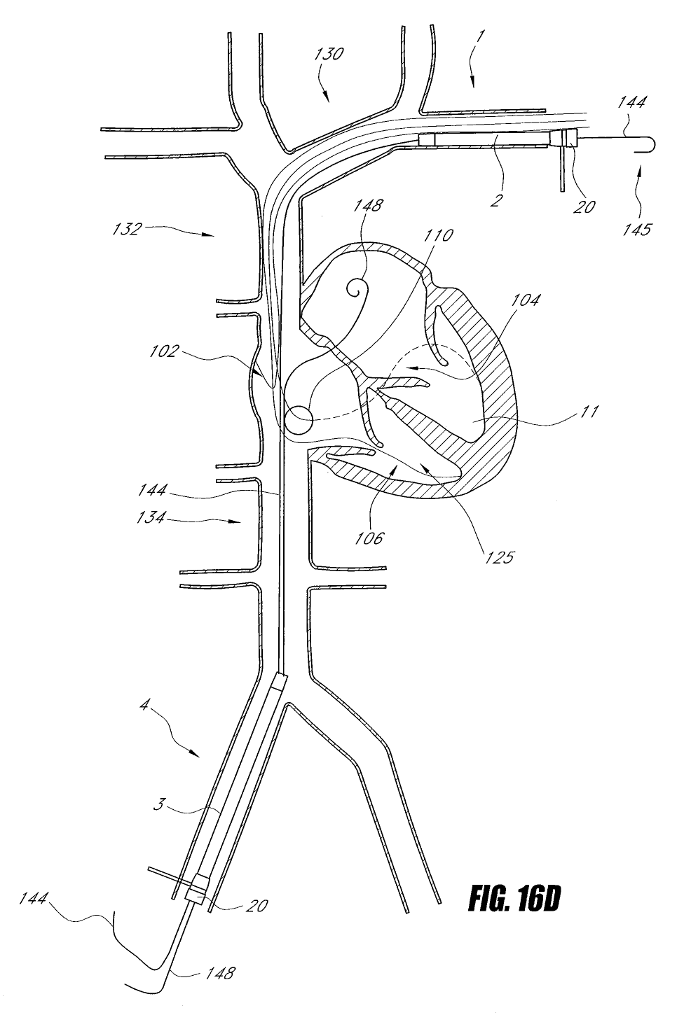

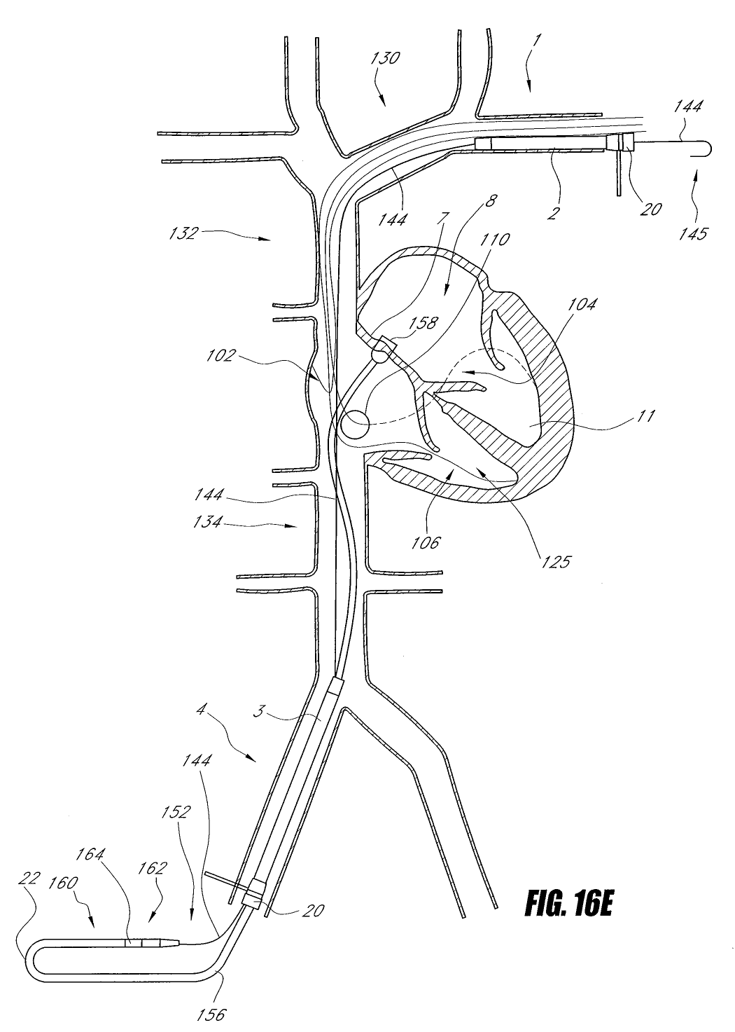

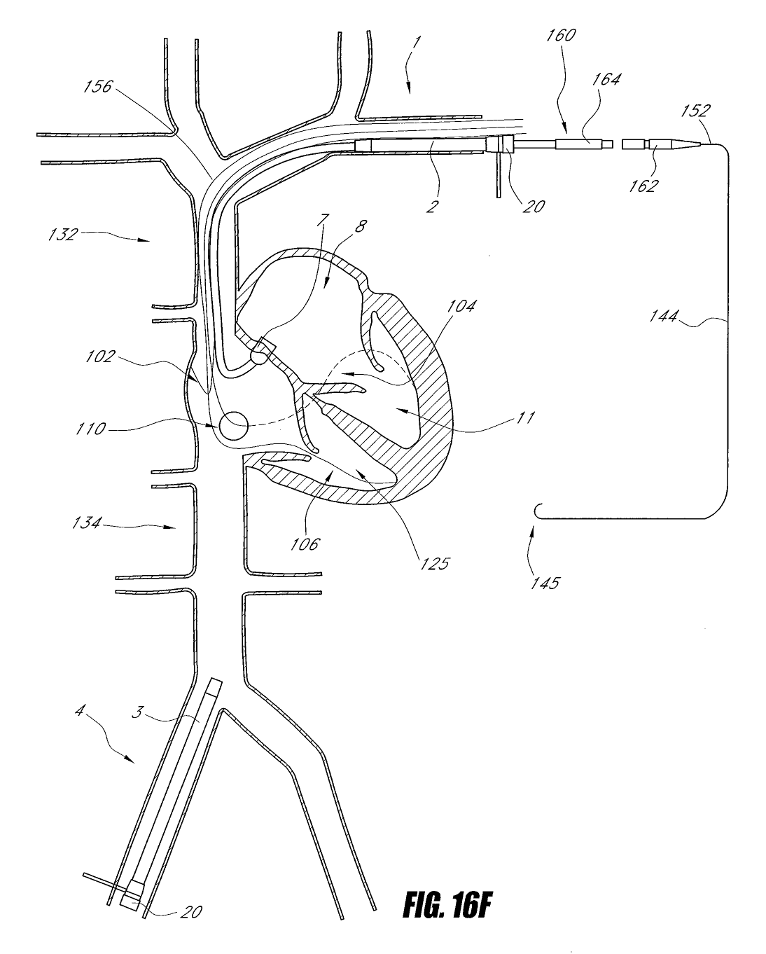

FIG. 16A schematically depicts the relationship between the cardiac anatomy and implanted leads with the venous vascular system. Because of the typical location of the CRM housing 126, the CRM leads 102, 104, and 106 are inserted into the venous vasculature along a pathway from the left subclavian vein 1, through the inominate vein 130, along the superior vena cava 132 and into at least the right atrium 9. As mentioned previously, access to the cardiac structures may also occur from the femoral veins, preferably the right femoral vein 4, which then empties into the inferior vena cava 134 through the external and common iliac veins. In some instances, the left femoral vein may be used as an access site. Likewise, other cardiac target locations may include the pulmonary veins and arteries, the left and right ventricular outflow tracts, and various locations on the epicardium. One or more leads may have been placed earlier during the same procedure involving the transfer procedure, or may have been placed during a prior procedure. To perform the transfer procedure in a patient with pre-existing implanted components in the vasculature, access at the initial insertion site and the exit or delivery site is used. Access is typically provided by vascular sheaths 2, 3 as discussed previously, and preferably with vascular sheaths 2, 3 with hemostasis valves 20. In other embodiments, only one sheath, or even no sheaths, is used. In some transfer procedures, more than two sheaths may be used because some sheaths may be swapped out during the procedure, depending upon the particular component being passed through the sheath, or because more than two anatomical access sites are involved in the transfer procedure. The hemostasis valves may be of any type or construction known in the art, but preferably comprise a Tuohy-Borst type of adjustable seal which can reduce or eliminate the resistance relating to insertion of the leads, sensors, guidewires or other components passed through them or a self-sealing type of hemostasis valve to minimize blood loss or air ingress. The sheaths used for the procedure may have the same or different configuration or features. Typically, the sheath 3 inserted at the initial insertion site is larger in internal diameter than the sheath 2 placed at the exit site, as the former requires an internal diameter sufficient to permit passage of the loop formed by the folding back of the transferred component against itself or the folding back of the guidewire or pulling member in a hairpin fashion. The latter sheath 2 typically is smaller because the loop is straightened out as the transferred component. The degree of size difference, if any, between the two sheaths, may depend on the length of the transferred component or its other physical characteristics, such as its stiffness or bendability. In certain embodiments, the transferred component and its guidewire or pulling member may be pushed into the vasculature to coil to allow passage of the transferred component through the sheath without having the component fold over itself. Thus, in some instances, it may be preferred that the loop is formed within the larger or more compliant vasculature.

FIG. 16B depicts the insertion of a stabilizer sheath 136 through the vascular sheath 3 at the insertion site. The distal end 140 of the stabilizer sheath 136 has been inserted past the right atrium 9 and in the general vicinity of the superior vena cava 132 and/or inominate artery 130, but in other embodiments, the distal end 140 may be positioned at other locations. A pull wire 144 is inserted into the sheath 136 and passed through the distal end 140 of the sheath 136 so as to place the distal end 145 of the pull wire 144 into the subclavian vein 1 and the inominate artery 130. In some embodiments, the pull wire 144 has a size of about 0.35″ or 0.38″, but in other embodiments, the pull wire 144 or pull member may be smaller or larger depending upon the component to be implanted and/or the vascular sites accessed. A snare 146 is inserted through the vascular sheath 2 to capture the distal end 145 of the pull wire 144 and the snare 146 is used to pull the distal end 145 out of the sheath 2 located at the exit site.

With the pull wire 144 externalized at the exit site, the transfer guidewire may be inserted to the target location. Referring to FIG. 16C, the transseptal puncture is preferably performed through the mid port 142 of the stabilizer sheath 136 to a target location, such as the intraatrial septum 7. In other embodiments, puncture through or insertion into the interventricular septum or the outer wall of a heart chamber may be performed to access a different target location. Puncture of the septum 7 between the left and right atria 8, 9 may be performed using a puncture component 150 in conjunction with the sheath 136 as described in U.S. Patent Publication Nos. 2006/0074398, 2006/0079769, and 2006/0079787, herein incorporated by reference in their entirety, or using other puncture components, including any of those described herein. After the puncture, a transfer guidewire 148 is placed in the left atrium 8. In some embodiments, the transfer wire 148 is preferably a LaCrosse 0.025″ wire, or Inoue 0.025″ (Toray) but in other embodiments, the wire used may have a different diameter, a different stiffness or other features. Using the stabilizer sheath 136 to position the guidewire 148 in the region with previously placed leads 102, 104, 106 permits the transfer guidewire 148 to take a path similar to the transseptal puncturing component 150, thereby making it less likely that the leads 102, 104, 106 will tangle when the proximal implantable sensor lead is repositioned.

Referring to FIG. 16D and FIG. 16E, after insertion of the transfer wire 148 to the target location, the stabilizer sheath 136 (not shown) is removed leaving the proximal ends 152, 154 of the pull guidewire 144 and the transfer wire 148, respectively, exiting the femoral venous sheath 3. The implantable sensor lead delivery sheath is then advanced over the transfer wire 148 into the left atrium 8. The implanted lead 156 is deployed with its sensor housing 158 fixed about the atrial septum 7 or other structure as appropriate. In certain embodiments, the transfer wire 148 may be removed before or after the insertion of the sensor lead delivery sheath. In other embodiments, the implanted lead 156 may be configured for insertion over the transfer wire 148, either with or without the delivery sheath. As described above, the transmembrane puncture may be performed after the externalization of the pull guidewire 144 by the snare 146, but in other embodiments the transmembrane puncture may be performed before the externalization of the pull guidewire 144.

FIG. 16E depicts the implanted lead 156 sensor housing 158 affixed to the atrial septum 7 such that the lead's distal sensor 158 is exposed to the left atrium 8. The proximal end 160 of the implanted sensor lead 156 is coupled to the pull guidewire 144. In one preferred embodiment, the pull guidewire 144 and the proximal end 160 of the sensor lead 156 are coupled using a coupler 162 that is configured to fasten to the proximal end 152 of the pull wire 144 and the proximal end 160 of the sensor lead 156 to facilitate a temporary joining of the two components. The coupler 162 can have any of a variety of configurations for temporarily joining the two components during the transfer procedure. Some embodiments of the coupler are described in greater detail below. In other embodiments, the two components may be joined together by other means, including suturing, gluing, friction, or taping

The coupling of the proximal end 152 of the pull wire 144 and the proximal end 160 of the sensor lead 156 forms a loop 22 external to the vascular sheath 3. As the distal end 145 of pull member 144 is pulled from the vascular sheath 2 located at the exit site, the proximal end 152 of pull wire 144 will pull the coupler 162 and the proximal end of the implantable sensor lead 156 into the insertion site sheath 3. The turn or loop 22 formed by the guidewire 144, coupler 162 and/or implantable sensor lead 156 will then pass through the hemostatic valve 20 of the vascular sheath 3. The turn 22 will move progressively more distally relative to the sheath 3 at the insertion until the turn 22 passes the target location and begins to unfold and straighten out. The proximal end 160 of the sensor lead 156 will then be pulled through the vasculature until it exits the circulation through the vascular sheath 2 in the subclavian vein 1 or other exit site. Preferably, the implantable sensor lead 156 body has sufficient flexibility to reduce the tension or force exerted on the distal sensor 158 during the procedure that may adversely affect the performance or calibration of the sensor 158. FIG. 16F shows the final configuration of the implantable sensor lead 156 after its proximal end 160 is pulled through the vasculature and out of the vascular sheath 2 at the exit site. The lead connector 164 at the proximal end 160 of the sensor lead 156 can then be attached to the device housing 126 (FIG. 15).

FIG. 17 shows one embodiment of a stabilizer sheath 136. The embodiment depicted generally in FIG. 17 is described in greater detail in U.S. Patent Publication Nos. 2006/0074398, 2006/0079769, and 2006/0079787, hereby incorporated by reference in their entirety. One of skill in the art will understand that other sheaths, sleeves or coverings may also be used. Briefly, the sheath 136 typically, but not always, has tubular a configuration, a proximal end 138, a distal end 140, and one or more side or mid-ports 142. The sheath 136 may further include a guide catheter that is slidable within a lumen of the sheath, the guide catheter including a tissue penetration member that may be used to achieve access within or through various tissue structures to reach the desired target location. The tissue penetration member preferably includes a guidewire lumen that may be used to pass a guidewire to the target location after tissue penetration. In other embodiments, however, the tissue penetration member may be withdrawn from the tissue and guidewire or other component may be placed through the tissue pathway formed by the penetration member. The sheath 136 may optionally have a monorail rapid exchange guidewire lumen located about the distal end 140 of the sheath 136. In other embodiments, the guidewire lumen may extend along the entire length of the stabilizing sheath 136 or a substantial portion thereof. The sheath 136 may be optionally configured with one or more intravascular ultrasound transducers or other imaging component about the distal end or side port(s) of the sheath 136, or associated with the guide catheter.

Although the pull wire 144 and proximal end 160 of sensor lead 156 may be joined during the transfer procedure using any of a variety of methods or devices known in the art, FIGS. 18A to 18C depict one preferred embodiment of a coupler 162 that may be used to join the components for the transfer procedure. The coupler 162 includes a main body 166 with caps 168, 170, located ends 172, 174 each configured with a cavity 176, 178 to retain the joined components. Each cavity 176, 178 is surrounded or formed by a collet structure 180, 182 comprising slots 184 and compression members 186. The cavities 176, 178 may have a length similar to the slots 184, or may extend farther centrally into the main body 166. The extension of the cavities 176, 178 may be equal or different. The collet structures 180, 182 can compress or clamp around the guidewire or implantable component inserted into the cavities 176, 178 and secure them together. The caps 168, 170 are configured to form an interfit around the respective coupler end 168, 170 that reversibly compress the compression members 186 into the cavities 176, 178 to secure the inserted component. The caps 184, 186 and/or main body 166 of the coupler 162 may be provided with a textured surface 188 or other reduced slip surface to facilitate engagement or disengagement of the caps 184, 186 and main body 166.

The main body 166 of the connector 162 is depicted in FIGS. 19A and 19C, without the caps 168, 170. Although the collet structures 180, 182 in FIGS. 19A and 19C are depicted with four slots 184 and four compression members 186, the collet structures 180, 182 may have a fewer or a greater number of slots 184 and compression members 186. For example, in other embodiments, the collet structure may comprise opposing two compression members that function like a clamp or vise. The collet structures at each end of the connector may have a different size or configuration, depending upon the particular component to be connected to any given end 172, 174 of the coupler. The compression members 186 may optionally have lips, flanges or surface texturing on their interior surfaces or to further reduce the risk of slippage out of the cavities. In still other embodiments of the invention, other interfit configuration between a component and an end of the coupler may be provided, including but not limited to complementary snap fits, friction fits, clamshell clamp configurations, eyelets, suturing retaining posts or apertures, etc. In one embodiment, the coupler 162 and the pull wire 144 (FIG. 16E) are integral such that the coupler 162 only requires one connector end with a locking cap for attaching the sensor lead 156. In a preferred embodiment, the pull wire 144 comprises a 0.035″ or 0.038″ guidewire and is manufactured with the coupler 162 as a single-piece device or is provided pre-attached from the manufacturer.

In other embodiments, the connector end of the coupler 162 may be configured to form a complementary interfit with the lead connector configuration of the implantable lead 156 and does not require a locking cap. Such lead connector configurations may include but are not limited to AO (American Optical Special), Bay (Biotronik Special/Bayonet), CCS (Cardiac Control Systems Special), ED (Edwards Special), GE (General Electric Special), IS-1 (3.2 mm connector, short pin, sealing rings), PSI (Pacesetter Special), SE (Siemens Special Threaded), TRI (Sorin Tripolar), and any of a variety of 3.2 mm, 5 mm and 6 mm lead connectors known in the art. Embodiments of the coupler comprising complementary connector interfit configurations may be preferred in the embodiments where the pull wire 144 is integrated with the coupler 162. In further embodiments, known lead connectors may be modified to facilitate the lead transfer process without affecting the integrity of the connection to the pacemaker/defibrillator or other implantable unit. For example, the inner wall of a hollow proximal IS-1 pin on an implantable lead could be configured with screw threads or other retaining features, and the proximal end of the pull wire had mating screw threads, or other features that reversibly mated with the proximal pin.

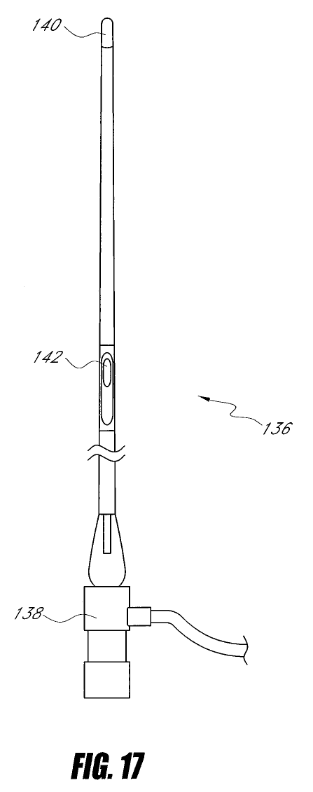

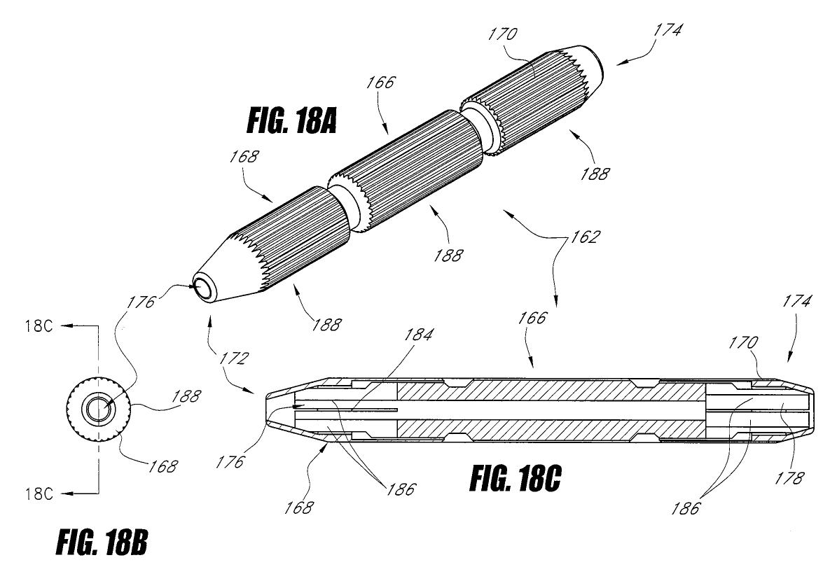

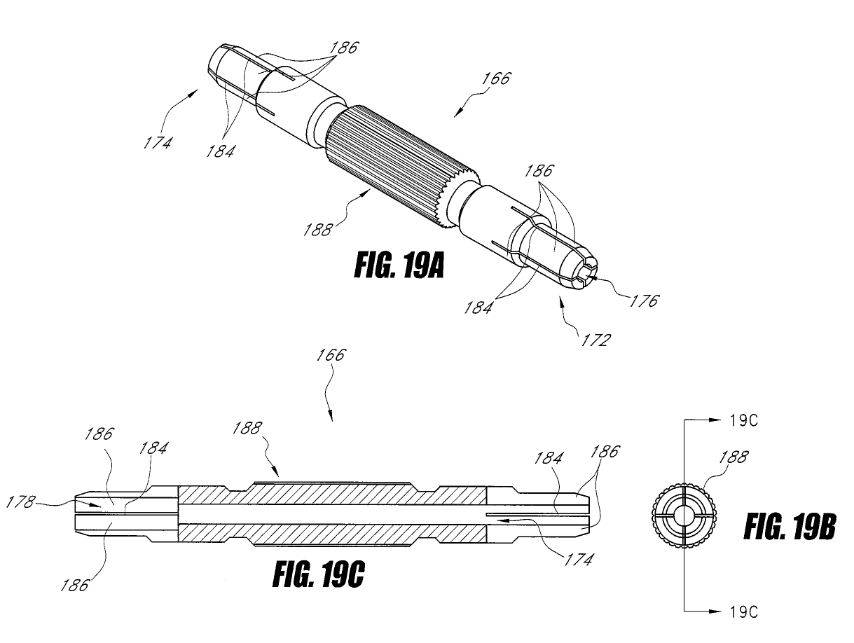

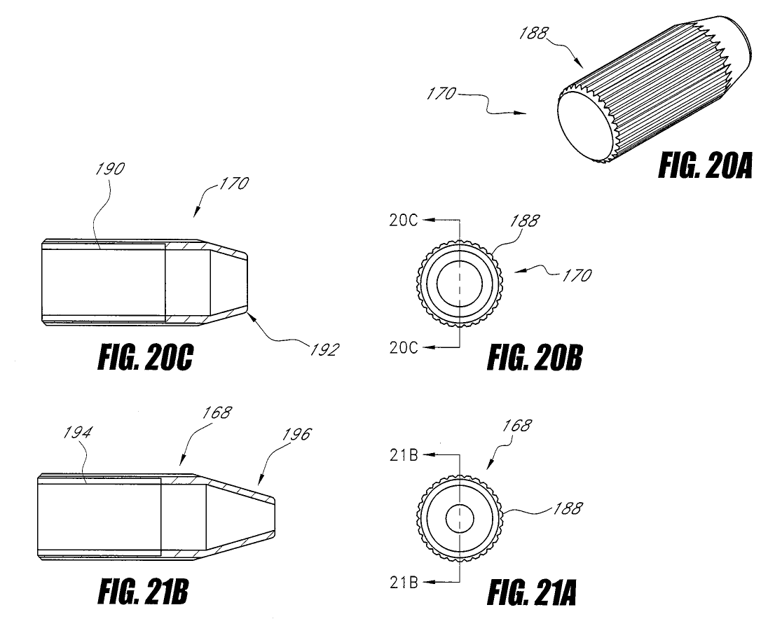

FIGS. 19A to 19C depict the main body of the coupler device of FIGS. 18A to 18C. FIGS. 20A to 20C and 21A and 21B depicted the locking caps 168, 170 usable with the main body 166 of FIGS. 19A to 19C. In this particular embodiment, the locking caps 168, 170 of the coupler 162 are depicted with different sizes and configurations because the shorter cap 170 with the larger internal diameter 190 and shorter taper region 192 is configured to retain the proximal end 160 of the sensor lead 156, which typically but not always has a larger outer diameter. On the other hand, the other cap 168 is configured with a smaller internal diameter 194 and a longer taper section 196 to cause greater movement of the compression members on the body to increase the clamping action onto the smaller outer diameter pull wire. To retain the locking caps 168, 170 on the main body 166 (FIG. 19A), any of a variety of interfits may be used between the inner surface of the caps 168, 170 and the outer or complementary surfaces of the main body 166. These interfits include but are not limited to complementary helical threads, complementary snapfits or interlocking fits.

In some embodiments, the coupler has an average diameter of about 0.08″ to about 0.20″ preferably about 0.11″ to about 0.14″ or most preferably about 0.12″ to about 0.13″ The connector preferably has a length of about 0.6″ to about 3.0″ preferably about 0.8″ to about 2.0″ or most preferably about 0.9″ to about 1.1″. The size and shape of the connector may vary according to the particular use, organ system or body lumen or cavity. The coupler may comprise any of a variety of biocompatible materials known in the art, including metals and polymers.

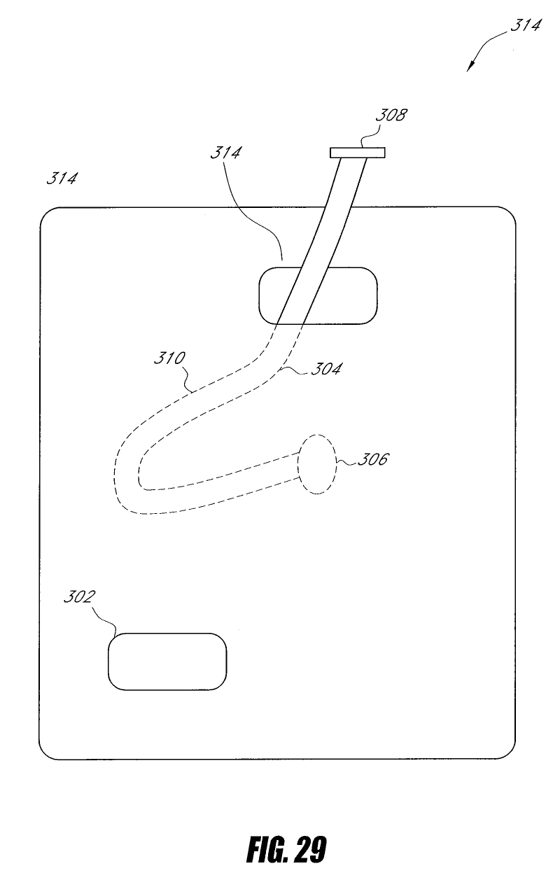

In another embodiment, a transfer guidewire is provided to transfer the proximal end of an implantable elongate component, such as a guidewire, lead, etc., from a first location to a second location. One such transfer guidewire is illustrated in FIG. 22. The illustrated transfer guidewire 200 includes a guidewire 202 that is unattached at its proximal end 204. The distal end 206 of the guidewire 202 is attached to a coupling 208, which in some embodiments is a rotational coupling assembly. In another embodiment, such as described below with respect to FIGS. 31-36, an interference fit coupling is provided. The coupling 208 is secured to the guidewire 202 with an end sleeve 210, which can be crimped, compressed, welded, adhered, or otherwise attached to the distal end 206 of the guidewire 202. The coupling 208 also includes a handle 212 and screw 214 to facilitate removable attachment of the coupling 208 to an elongate member, such as a lead, as described below. The handle 212 includes an atraumatic, or rounded, surface to facilitate pulling of guidewire 202 through the patient's body without causing damage to the organs or lumens through which it is pulled, as discussed below. A stylet 216 extends from the distal end of the transfer guidewire 200. In one embodiment, the stylet 216 is made from nickel-titanium (e.g., Nitinol). The stylet 216 can have the form of a solid wire. In one embodiment, the stylet 216 provides stiffness and increased column strength to the lead into which it is inserted, and allows the clinician to push the elongate body into which it is inserted into the patient's body, as described below.

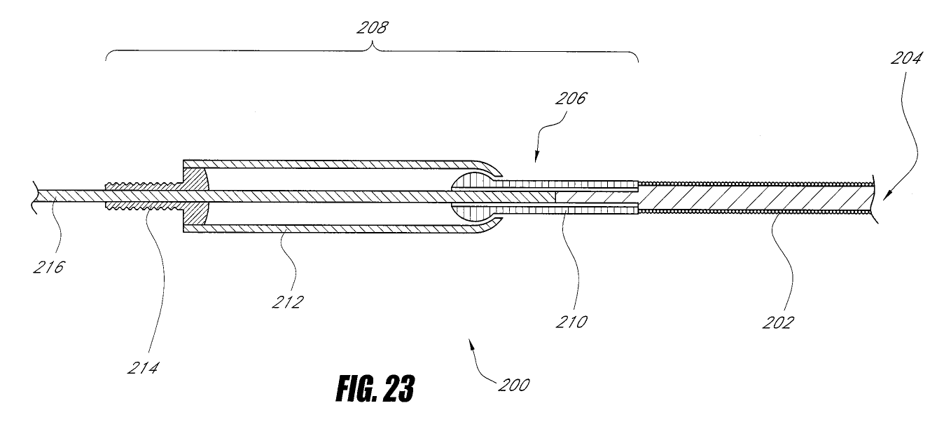

A cross-sectional view of a portion of the transfer guidewire 200 is illustrated in FIG. 23. In one embodiment, the guidewire 202 is hollow and is attached to an end coupling 210 at the guidewire 202 distal end 206. The end coupling 210 can include any of a variety of components that allow the handle 212 to be rotated or spun with respect to the guidewire 202, while limiting axial movement of the handle 212 with respect to the guidewire 202. For example, as illustrated in the embodiment of FIG. 23, the end coupling 212 can include a bulbous portion that has a larger diameter that the opening of the handle 212. Other mechanisms, such as ball joints, rings, etc., may be used to rotationally couple the guidewire 202 to the handle 212 and form a coupling 208.

As illustrated in FIG. 23, the screw 214 is secured to the handle such that handle rotation causes the screw 214 to turn. The screw 214 includes an opening or lumen that receives the stylet 216. The screw 214 lumen typically has a slightly larger diameter than the stylet so the screw 214 may rotate with respect to the stylet without binding or otherwise rubbing against the stylet 216. In one embodiment, the stylet 212 is attached to the guidewire 202 at the end coupling 210. The stylet 212 passes through the opening in the screw 214 and an opening in the end of the end coupling 210 and is crimped, compressed, welded, adhered, or otherwise attached to the end coupling 210 and guidewire 202. When the transfer guidewire 200 is assembled, the handle 212 and screw 214 may be rotated with respect to the stylet 216, end coupling 210, and guidewire 202.

The rotational coupling 208 advantageously provides simpler, safer, quicker mechanism for intra corporeal lead transfer. For example, the transfer guidewire 200 may be used in accordance with any of the lead or guidewire transfer methods described herein. For example, with respect to FIGS. 16A-F (particularly FIG. 16B), instead of advancing a pullwire 144 into the sheath 136 and advancing it through the distal end 140 of the sheath 136 so as to place the distal end 145 of the pull wire 144 into the subclavian vein 1 and the inominate artery 130, a transfer guidewire 200 may be used. For example, in one embodiment the guidewire 202 of the transfer guidewire 200 is inserted into the sheath 136 and advanced superiorly until it extends past the distal end 140 of the sheath 136 and into the subclavian vein 1 and the inominate vein 130. The end of the guidewire 202 can include a j-tip or other feature to allow it to be easily retrieved with a snare 146. In other embodiments, a sheath 136 is not used to advance the transfer guidewire 200 superiorly. Instead, the guidewire portion 202 of the transfer guidewire 200 is steered superiorly through the vasculature using visualization techniques. The end of the transfer guidewire 200 is retrieved with a snare 146 and pulled superiorly until it is externalized at the snare insertion point. In another embodiment, the transfer guidewire 200 is inserted into the body at a superior location, and advanced inferiorly until its stylet 216 and rotational coupling 208 exit the body at a second, inferior location.

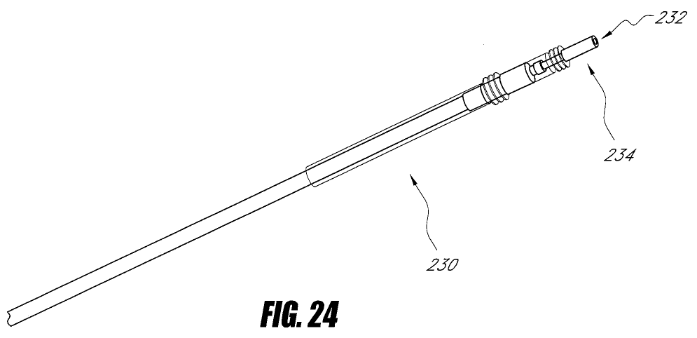

One embodiment of an implantable sensor lead that may be used as the implanted lead 156 of FIG. 16E is shown as sensor lead 230 of FIG. 24. The sensor lead 230 includes a sensor housing (not shown) at the lead 230 distal end (not shown). The sensor lead 230 also includes an opening 232 at a threaded connector 234 located at the lead's proximal end. The connector 234 is configured to receive and mate with the threaded screw 214 of the transfer guidewire 200.

For example, once the sensor lead 230 is delivered to the atrial septum of the patient's heart (for example, as described above with respect to the implantation of the sensor lead 156 of FIGS. 16A-F), the transfer guidewire 200 stylet 216 is inserted into the opening 232 at the threaded connector 234. The stylet 216 is advanced into a lumen of the sensor lead 230 until the screw 214 engages the threaded connector 234 at the lead 230 proximal end. The rotational coupling 208 is rotated to spin the screw 214 with respect to the guidewire 202 and stylet 216, and to attach the guidewire assembly 200 to the sensor lead 230. FIG. 25 shows the transfer guidewire 200 attached to the sensor lead 230, in such manner.

Once attached, the handle 212 and screw 214 of the transfer guidewire 200 is rigidly attached to the sensor lead 230, but the guidewire 202 and stylet 216 may be rotated with respect to the sensor lead 230. Such configuration advantageously allows the proximal end of the sensor lead 230 to be drawn superiorly through the patient's vasculature without providing torque or twisting of the sensor lead 230. Such torque or twisting could be transferred to the sensor 158 secured to the atrial septum 7, which could cause it to become dislodged, misaligned, or miscalibrated. The rotational coupling assembly 208 helps avoid these potentially serious complications.

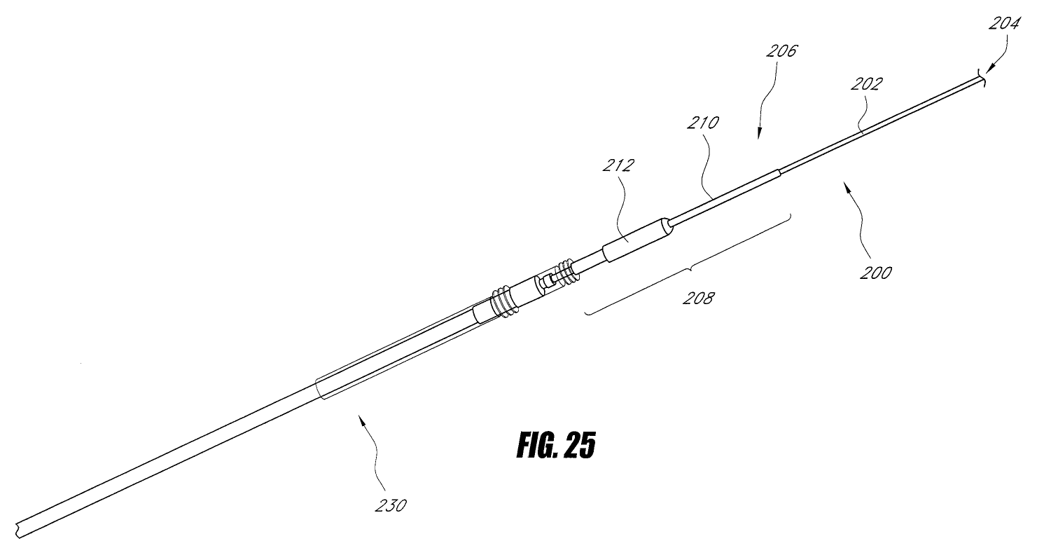

FIG. 26 illustrates a cross sectional view of a portion of the attached sensor lead 230 and transfer guidewire 200, as described above. FIG. 26 shows the stylet 216 extending into a sensor lead central lumen 236 through electrical contacts 238 located near the sensor lead's 230 proximal end. Once attached, and after the guidewire 202 of the transfer guidewire 200 has been externalized, the guidewire 202 may be pulled away from the patient's body in order to externalize the proximal end of the sensor lead 230 at the desired location.

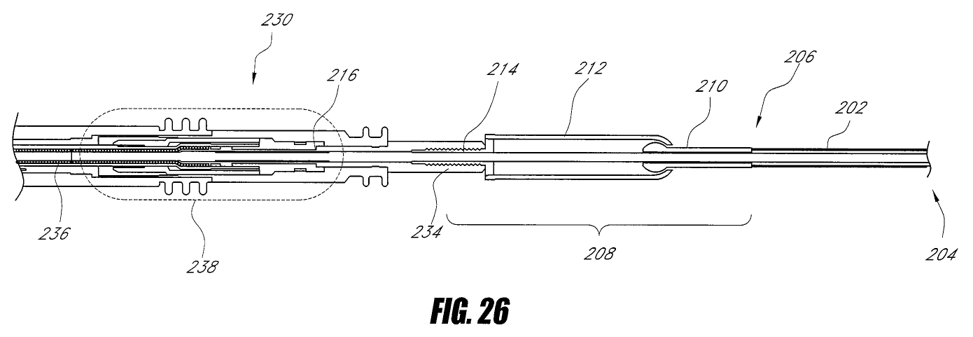

FIGS. 27-29 illustrate another embodiment of a lead transfer procedure that may be performed with any of the devices described herein, including the transfer guidewire 200 of FIGS. 22-26. Devices and methods of various embodiments are used to transfer the externalized end of an implanted elongate body from a first access point to a second access point. In one embodiment, the transfer is accomplished without disturbing, moving, dislodging and/or affecting the second, implanted end of the flexible elongate body.

FIG. 27 illustrates a schematic view of a patient's body 300. A first elongate body 304 has been partially inserted into the patient's body 300 at a first access point 302. The first elongate body 304 includes a first end 306, a second end 308, and a flexible body portion 310 extending between the first and second ends 306, 308. The first end 306 has been positioned at a desired location within the patient's body 300. For example, in some embodiments, the first end 306 is positioned within the patient's heart, atrial septum, ventricle, or any other location within the medical patient.

FIG. 28 shows a second elongate body 312 that has also been inserted into the patient's body 300. The second elongate body 312 extends between the first access point 302 and a second access point 314. The second elongate body 312 includes a first end 316, a second end 318, and a flexible body portion 320 extending between the first and second ends 316, 318. In one embodiment, the second elongate body 312 is inserted into the patient's body 300 at the first access point 302 and advanced to the second access point 314. In another embodiment, the second elongate body 312 is inserted into the patient's body 300 at the second access point 314 and advanced to the first access point 302.

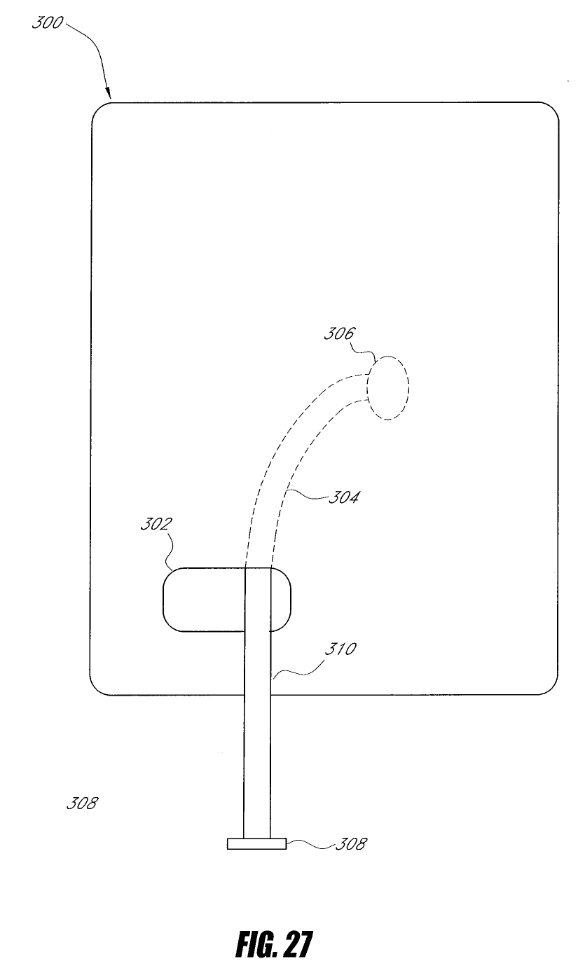

The first end 316 of the second elongate body 312 resides outside of the patient's body 300 at the first access point 302. The first end 316 of the second elongate body 312 is attached to the second end 308 of the first elongate body 304. Once attached, the second elongate body 312 is withdrawn from the patient's body 300 via the second access point 314. As the second elongate body 312 is withdrawn, the second end 308 of the first elongate body 304 is withdrawn through the patient's body, as well. When the second end 308 of the first elongate body 304 reaches (or is externalized at) the second access point 314, the first and second elongate bodies 304, 312 are disconnected from each other. Once disconnected, the second end 308 of the first elongate body 304 is positioned at or near the second access point 314, as shown in FIG. 29.

The first and second access points 302, 314 can include any of a variety of locations for entering the patient's body 300. For example, in some embodiments, the first and second access points 302, 314 include a vein, an artery, a bodily lumen, a bodily cavity, an air passage, a portion of the digestive tract, a femoral vein, a subclavian vein, the nose, the mouth, and/or other bodily location. The first and second access points 302, 314 may also include any of the access points described herein.

The first elongate body 304 includes any of a variety of devices for entering and/or treating the patient's body 300. For example, in some embodiments, the first elongate body 304 includes an implantable lead, an electrode, conductors, a tube, a cannula, a catheter, an aspiration line, a wire, and/or a guidewire. The first elongate body 304 may also include any of the devices described herein.

The first end 306 of the first elongate body 304 can include any of a variety of devices for treating, measuring, and/or manipulating the patient's body 300. For example, in some embodiments, the first end 306 includes an electrode, a sensor, an anchor, a clamp, a sensor, a pressure sensor, and/or a thermometer. The first end 306 may also include any of the devices described herein.

The second end 308 of the first elongate body 304 can include any of a variety of devices for coupling to the first elongate body 304 and/or treating, measuring, and/or manipulating the patient's body 300, as well. For example, in some embodiments, the second end 308 includes a connector, a header, a pacemaker connector, a CRM connector, an antenna connector, a joint, a ball joint, a rotational coupling, a swivel, and/or a clip. The second end 308 may also include any of the devices described herein.

The flexible body portion 310 can include any of a variety of devices for treating, measuring, and/or manipulating the patient's body 300. For example, in some embodiments, the flexible body portion 310 includes a lead, wires, a tube, a cannula, a catheter, and/or an aspiration line, etc. The flexible body portion 310 may also include any of the devices described herein.

The second elongate body 312 includes any of a variety of devices for entering and/or treating the patient's body 300. For example, in some embodiments, the second elongate body 312 includes a guidewire, a transfer guidewire, as well as any of devices used with the first elongate body 304, including an implantable lead, an electrode, conductors, a tube, a cannula, a catheter, an aspiration line, and/or a wire. The second elongate body 312 can also include a stiffening member, such as a reinforced portion, a stylet, etc. The second flexible body 312 may also include any of the devices described herein.

The first end 316 of the second elongate body 312 can include any of a variety of devices for coupling to the second elongate body 312 to the first elongate body 304. For example, in some embodiments, the first end 316 includes a connector, a header, a pacemaker connector, a CRM connector, an antenna connector, a joint, a ball joint, a rotational coupling, a swivel, and/or a clip. The first end 316 may also include any of the devices described herein.

The second end 318 of the second elongate body 312 can include any of a variety of devices for manipulating the second elongate body 312. For example, in some embodiments, the second end 318 includes a guidewire, a pullwire, a wire, a tube, a line, a cable, etc. The second end 318 may also include any of the devices described herein.

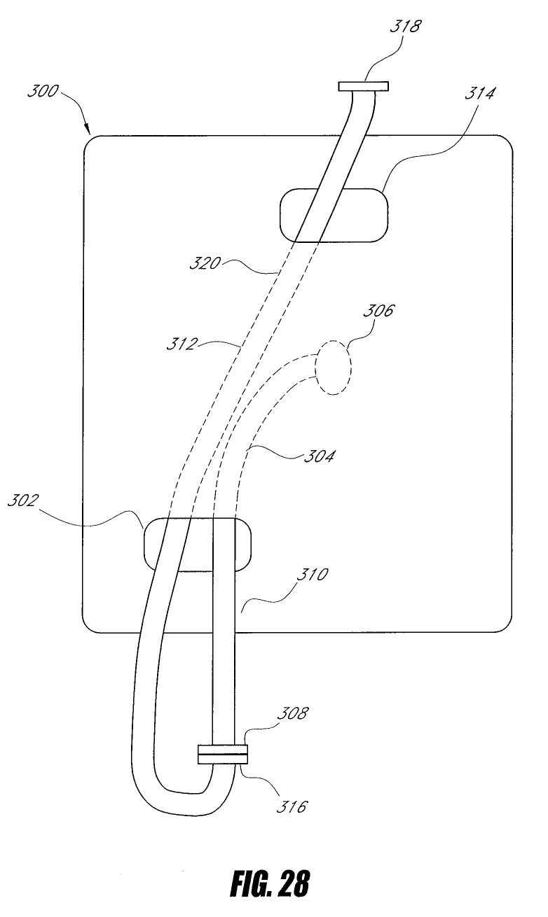

FIG. 30 illustrates one embodiment of a method of manipulating an implanted flexible elongate body. The method 400 begins at block 402. At block 402, a first implantable flexible elongate body is implanted within a medical patient via a first access point. The first implantable flexible elongate body can include a lead, or any of the elongate bodies described herein. At block 404, a second flexible elongate body is provided to the patient's body such that the second flexible elongate body extends between the first access point and a second access point. The second flexible elongate body can include a transfer guidewire, or any flexible elongate body described herein. In another embodiment, the method 400 begins at block 404, proceeds to block 402, and then to block 406, as the order of at least blocks 402 and 404 are interchangeable.

At block 406, the ends of the first and second flexible elongate bodies at the first access point are secured to each other. In one embodiment, at least one of the ends includes a swivel or rotational decoupler such that the first and second elongate bodies may be rotated with respect to each other. At block 408, the second flexible elongate body is withdrawn from the patient via the second access point until the attached end of the first flexible elongate body reaches the second access point or exits the patient via the second access point.

At block 410, the first and second elongate bodies are disconnected from each other. At optional block 412, the end of the second elongate body is attached to a device at or near the second access point.

Another embodiment of a transfer guidewire is illustrated in FIGS. 31-36. The transfer guidewire 430 of FIGS. 31-36 is similar to the transfer guidewire of FIGS. 22-26. The transfer guidewire 430 of FIGS. 31 and 32 includes a catheter 432 formed at it proximal end 434, and a guidewire 436. The guidewire 436 has a curve, or bend 438, such as a J-tip, at the guidewire's distal end 440. The bend 438 allows the transfer guidewire 430 to be snared from within a patient's body, as described in detail below.

The catheter 432 at the guidewire 430 proximal end 434 has a chamber or lumen 446 that is sized to form an interference fit over the outside diameter of an implantable elongate body, such as a lead, pacemaker lead, catheter, cannula, tube, wire assembly, cable, etc. The catheter 432 has an outside wall 442 and an inside wall 444, which defines a chamber 446. The chamber 446 and inside wall 444 are sized to form an interference fit with an elongate body to which the transfer guidewire 430 is to be attached. In one embodiment, the interference fit is designed to keep the transfer guidewire 430 and elongate body to which is attached coupled together when at least 1.5 times the maximum pull force provided during a transfer procedure, as described herein.

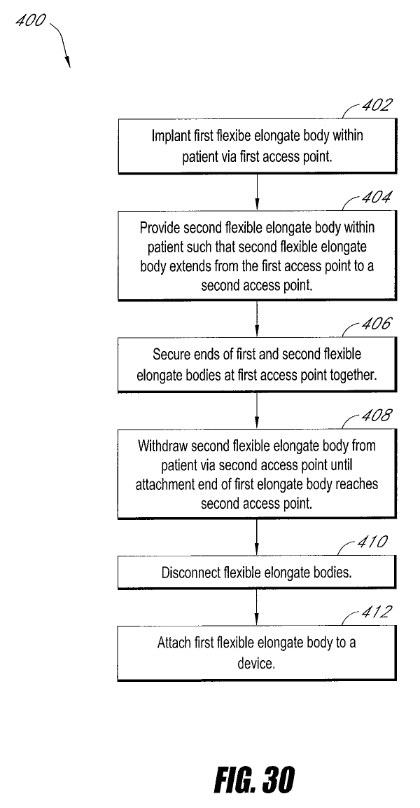

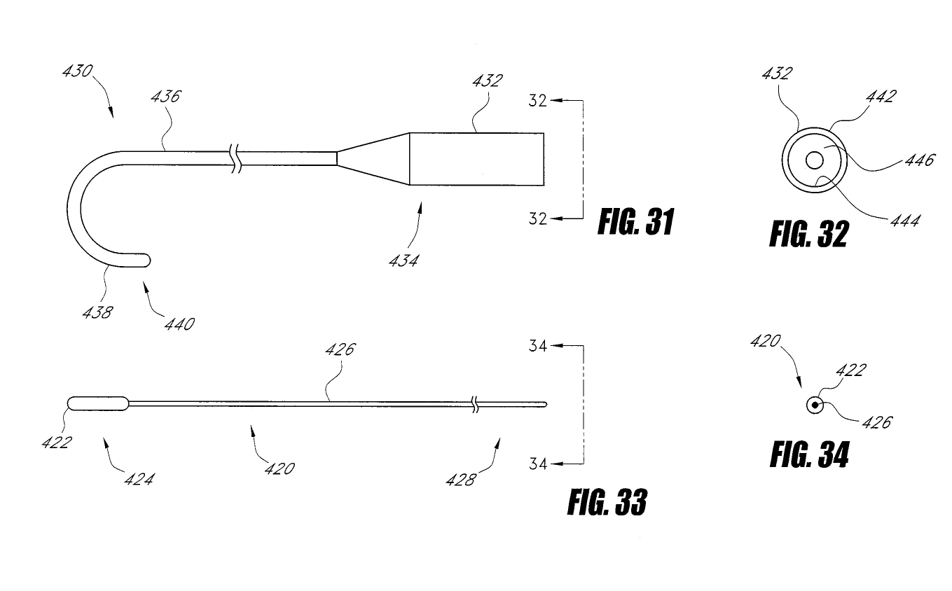

The cavity 446 is also configured and sized to receive the proximal end of a stylet. A stylet, such as the stylet 420 illustrated in FIGS. 33 and 34, can be inserted into the end of the flexible elongate body prior to transfer. The stylet 420 provides additional stability to the flexible elongate body, and facilitates pushing and/or pulling of the flexible elongate body through a patient's body, including the patient's vasculature.

The stylet 420 includes a handle 422 at its proximal end 424 and a wire 426 extending to the stylet's distal end 428. The stylet's wire 426 is preferably made from nickel titanium or another metal, alloy, or other material, that is flexible but will not kink when inserted into a patient's body.

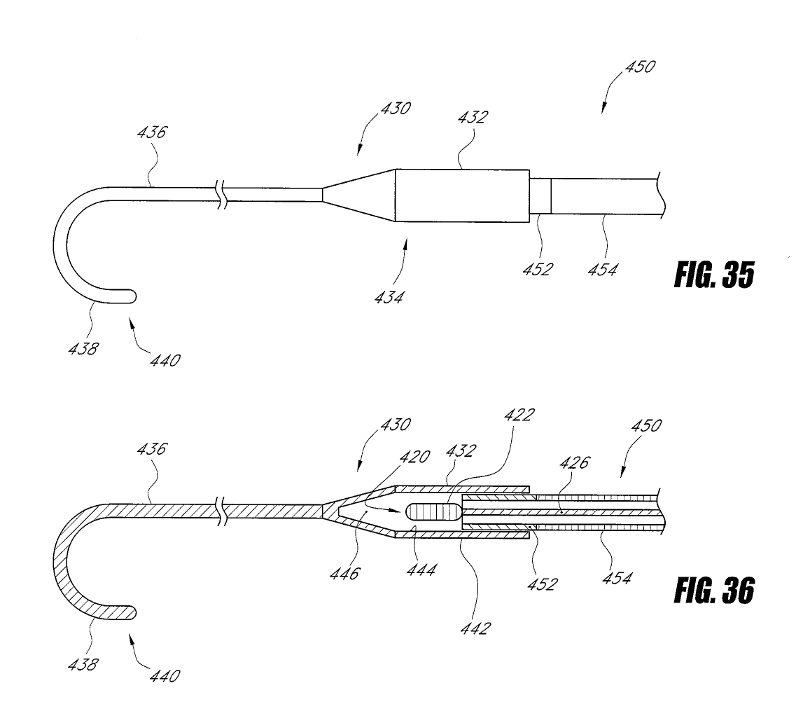

FIGS. 35 and 36 show a flexible elongate body 450 that has been attached to the transfer guidewire 430 and stylet 420 of FIGS. 31-34. The flexible elongate body 450 of FIGS. 35 and 36 includes a lead 454 (or other tubular structure) that is extends to the elongate flexible body's proximal end 452. A transfer guidewire 430 is selected such that it's catheter cavity forms an interference fit with the proximal end 452 of the flexile elongate body 450. A stylet 420 is inserted into a lumen of the elongate body 450 prior to attachment to the transfer guidewire 430. A cross-section view of the elongate body 450, stylet 420, transfer guidewire 430 assembly is illustrated in FIG. 36.

The transfer guidewire 430, or other transfer guidewire described herein, can be used in one embodiment to transfer the proximal end of an implanted, flexible elongate body from first to second locations at the patient's body without removing its implanted distal end. For example, the transfer guidewire can be used to transfer the proximal end of an implantable sensor lead from a femoral location to a subclavian location.

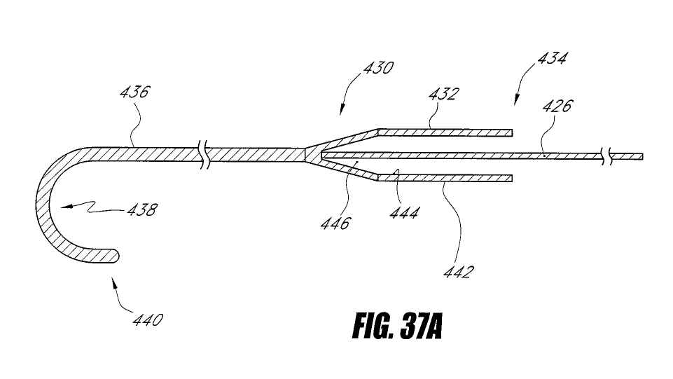

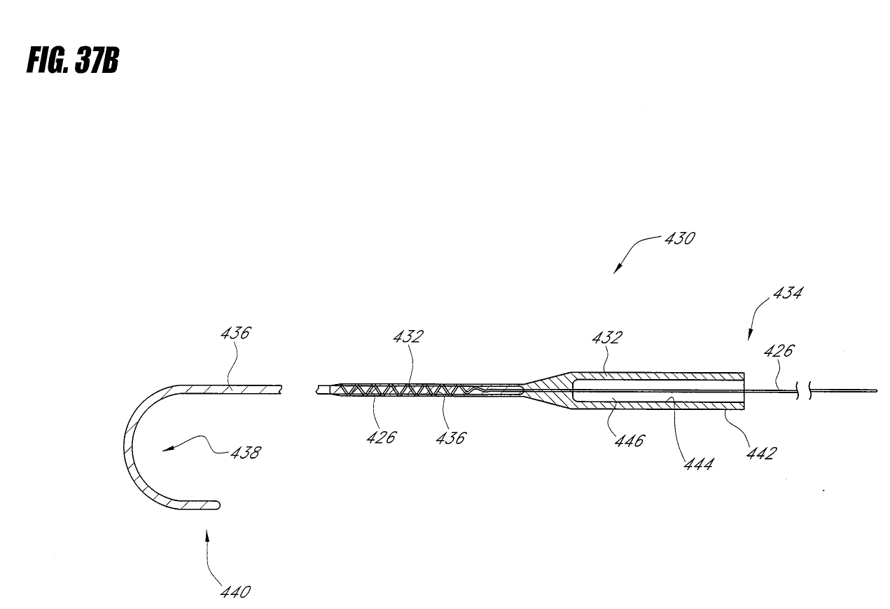

In yet another embodiment, a stylet 420 is integrated into the transfer guidewire 430 itself. For example, as illustrated in FIG. 37A, a stylet wire 426 is attached at its proximal end to the inside cavity 446 of the transfer guidewire's 430 catheter 432. For example, the transfer guidewire's 430 guidewire 436 and stylet 426 can be coaxially aligned. In addition, the guidewire 436 and stylet 426 are sometimes attached to each other at their respective proximal ends, and the catheter 432 is positioned around one or both of them. For example, in one embodiment, the guidewire 436 extends distally away from the catheter 432 and the stylet wire 426 extends proximally within the catheter chamber or lumen 446, and then exits the chamber 446, as shown in FIGS. 37A and B. The stylet 426 extends beyond the proximal end 434 of the catheter 432 a sufficient distance such that it is configured to be inserted into a channel of a flexible lead body 450.

In the embodiment illustrated as FIG. 37B, a stylet 420 is integrated into the transfer guidewire 430, as well. The stylet wire 426 of FIG. 37B is wrapped around the proximal end of a guidewire 436. The wrapped is surrounded by one end of the transfer guidewire's catheter 432. The end of the transfer guidewire's catheter 432 can be heated to flow around and secure the stylet wire 426 to the guidewire 436 proximal end. For example, in one embodiment, the catheter 432 is made of a flowable material, such as a plastic, a resin, a polyether block amide, a thermoplastic elastomer made of flexible polyether and a rigid polyamide, PEBAX®, or other similar material. The flowed end of the catheter 432 tapers outward to form the catheter's larger diameter proximal end 434. The stylet wire 426 exits a chamber 446 of the catheter's larger diameter proximal end 434, as discussed above with respect to FIG. 37A.

The following dimensions can apply to any one or more of the embodiments described herein. In some embodiments, the wrapped portion of the guidewire 436 is about 1″ or 2.5 cm long. In some embodiments, the guidewire is about 150 cm long. In some embodiments, the larger diameter proximal end of the catheter is about 10 cm long. In some embodiments, the stylet extends for about 55 cm from the end of the transfer guidewire catheter. In some embodiment, the guidewire has an outside diameter of about 0.038″ or about 9.6 mm. In some embodiments, the stylet wire's diameter is about 0.008″ or about 2 mm at the end that wraps around the guidewire, and about 0.014″ or about 3.6 mm in diameter at its opposite end. Furthermore, in some embodiments, the inside diameter of one end of the transfer guidewire's catheter is sized to receive and form an interference fit with the connector pin and/or proximal end of an implantable lead (e.g., a pressure sensor lead, cardiac pacing lead, etc.).

One method of transferring the proximal end of an implanted, flexible elongate body includes placing an introducer (e.g., a 16F introducer or other introducer) or other delivery catheter in the patient's femoral vein at a femoral vein insertion location, and a short delivery sheath in a subclavian vein at a subclavian vein insertion location. The implantable sensor and lead are percutaneously advanced to the patient's heart via the femoral vein insertion location. The patient's atrial septum is punctured, and access to the patient's left atrium is secured by using a guidewire such as a Toray guidewire. The opening to the septum is dilated with a dilator. Once dilated, a long delivery sheath is percutaneously advanced from the femoral vein insertion location to the dilated opening in the patient's septum. The distal end of an implantable sensor is advanced to the left atrium via the femoral insertion location through the long delivery sheath and anchored to the atrial septum. The distal end of the implantable sensor is coupled to an implantable lead at the lead's distal end. The proximal end of the implantable lead remains externalized with respect to the patient at the femoral insertion location.

The long delivery sheath is removed from the patient's vasculature and a stylet, such as a nitinol transfer stylet, or stylet 420 is inserted into a lumen of the implantable sensor lead. The proximal end of a transfer guidewire, such as the transfer guidewire 430 described above, is slipped over the proximal end of the stylet and implantable sensor lead. For example, in one embodiment, the proximal end of the transfer guidewire includes a catheter that forms an interference fit with the outside diameter of the implantable sensor lead's proximal end.

The distal end of the transfer guidewire is inserted into the patient via the introducer at the femoral insertion point, and advanced until its distal end reaches the patient's inferior vena cava. The distal end includes a J-tip end, or other feature that allows the transfer guidewire to be snared. A snare is inserted into the patient's vasculature at the subclavian insertion point. The snare is advanced inferiorly until it reaches the inferior vena cava. The snare is used to snare or otherwise attach to the J-tip end (or other feature) of the transfer guidewire. The snare is retracted superiorly through the patient's vasculature until it and the distal end of the transfer guidewire are externalized from the patient at the subclavian insertion location.

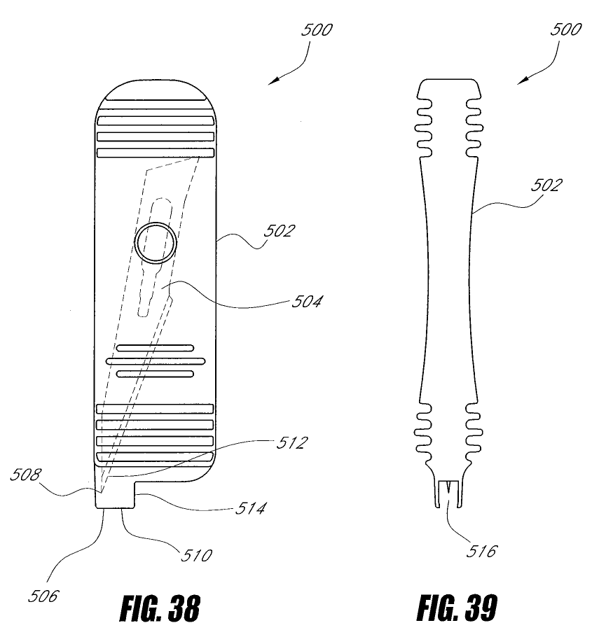

Lead transfer is performed by pulling or otherwise advancing the transfer guidewire in the superior direction. As the transfer guidewire is advanced superiorly the proximal end of the implantable sensor lead is also advanced superiorly, due to the connection between the transfer guidewire and the implantable sensor lead at their proximal ends. The implantable sensor lead is “pulled” superiorly until its proximal end is brought near, to, or past the subclavian access point. The proximal ends of the transfer guidewire and implantable sensor lead are separated. In one embodiment, the proximal end of the implantable sensor lead is attached to one or more implantable medical devices, such as a cardiac rhythm management device, pacemaker, defibrillator, pressure sensing system, etc. The proximal ends of the transfer guidewire and implantable sensor lead can be separated from one another by using a lead transfer slitter, as described below with respect to FIGS. 38 and 39.

One embodiment of a lead transfer slitter is illustrated in FIGS. 38 and 39. The lead transfer slitter 500 advantageously allows controlled decoupling of the catheter portion of a transfer guidewire (such as transfer guidewire 430) and the proximal end of an implantable lead. Controlled decoupling allows the catheter to be cut without damaging or cutting the implantable lead, its seals (e.g., o-rings, etc.), and without risk of cutting the user.

The lead transfer slitter 500 includes a housing 502 that contains a blade 504. The housing exposes just enough of the blade 504 to control cutting depth into the transfer guidewire catheter while shielding the blade 504 from the user. For example, in one embodiment the housing 502 includes a projection 506 in which an end portion of the blade 504 extends. The blade tip 508 is fixed a predetermined distance from the bottom edge 510 of the projection 506. In addition, the blade's cutting edge 512 is spaced a predetermined distance from the projection's leading edge 514.

The projection 506 defines a cavity 516 into which the catheter portion of a transfer guidewire is inserted. The transfer guidewire includes a catheter at its proximal end. An implantable elongate body, such as an implantable lead, is inserted into the catheter. As the lead transfer slitter 500 is drawn over the transfer guidewire, the blade 504 slices through the wall of the transfer guidewire's catheter without piercing or cutting the implantable, flexible elongate body, or lead. The catheter, and transfer guidewire, may then be separated from the lead.

Although certain procedures discussed above relate to a transseptal procedure wherein an elongate sensor component is inserted across the intra-atrial septum, the procedure may be adapted to any of a variety of body lumens, cavities or spaces, including other cardiac structures, the peripheral vascular system, the central nervous system, the gastrointestinal system, pulmonary system, urogenital system and other organ systems.

In one example, FIGS. 12A through 12C illustrate an embodiment of the invention adapted for the transfer of a gastric tube 40 from an oral first insertion site 41 to a nasal second insertion site 42. While an oral insertion site 41 is often a quicker and easier route for establishing a gastric 40 or endotracheal tube, a nasal insertion site 42 is usually more comfortable for the patient, particularly when the tube 40 must be left in place for extended periods of time, or when the patient is conscious. FIG. 12A shows the placement of a guidewire 43 from a second insertion site 42 through the nose, via a nasal sheath 44, to the first insertion site 41 in the mouth. In FIG. 12B, the distal end of the guidewire 43 is connected to the proximal end 45 of the gastric tube 40, and the guidewire 43 is withdrawn through the nasal sheath 44, pulling the proximal end of the gastric tube 40 back into the throat 46. FIG. 12C shows the final configuration of the gastric tube 40 after the complete withdrawal of the guidewire 43 and the nasal sheath 44, completing the transfer of the gastric tube insertion site from the mouth to the nose.

In another embodiment, the method of manipulating insertion pathways for accessing target sites further comprises providing a kit, or system, for performing the guidewire and/or medical device transfer. In one embodiment, the kit, or system, is a combination, assemblage and/or compilation of materials suitable for a common purpose and comprises an introducer sheath for each insertion site, a torqueable catheter and two guidewires. In another embodiment, the kit further comprises at least one of the guidewires having a coilable soft curled tip. In another embodiment, the kit further comprises at least one of the guidewires having a movable inner core mandrel. In another embodiment, the kit or system further comprises a snare. In another embodiment, the kit further comprises a thin-walled introducer. In a further embodiment, the kit includes a Brockenbrough needle catheter. In yet another embodiment, the kit further includes a Mullins sheath.

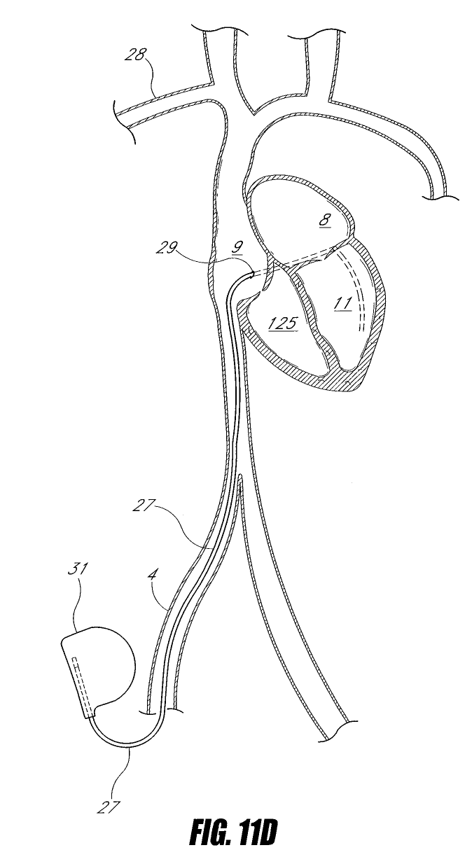

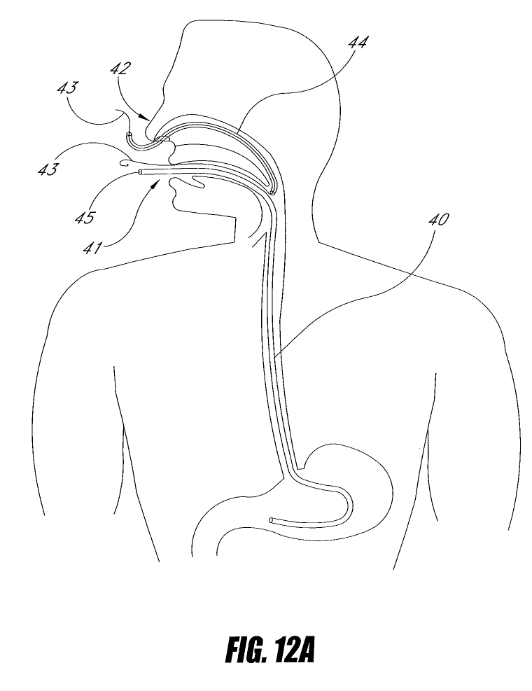

In another embodiment, a guidewire for manipulating insertion pathways to access target sites in the body is provided. In one embodiment, the guidewire 10 (FIG. 1) has a length of about 150 cm to about 350 cm, preferably between about 180 cm to about 280 cm, more preferably between about 220 cm to about 250 cm. In one embodiment, the guidewire 10 has an outer diameter of about 0.010 to about 0.064 inches. The outer diameter of the guidewire 10 need not be uniform throughout the length of the guidewire. In one embodiment, the distal portion 12 of the guidewire 10 may have a reduced diameter to facilitate insertion of the guidewire 10 into body structures or catheters. In another embodiment, changes to the diameter of the guidewire 10 along the length of the guidewire may also be used to alter the stiffness and flexibility along those portions. The guidewire 10 may be configured with a blunt distal end 34 (FIG. 13A) for reducing the risk of damaging tissue during manipulation of the guidewire 10. In another embodiment, the guidewire 10 features at least one radio-opaque marker (not shown) along the length of the guidewire to provide visualization of the guidewire under radiography or fluoroscopy.

As used herein, the term guidewire shall be given its ordinary meaning and shall include a wire positioned in, on, or through the body (for example, in, on, or through an organ, vessel, or duct) in order to direct the passage of another device over or along its length. The term pull wire as used herein shall be given its ordinary meaning and shall also include guidewires, polymeric and metallic sutures, snares or other elongate structures that may be used to push, pull, twist or otherwise manipulate a device.

Guidewires may be configured as single piece or multi-piece constructions. In one embodiment, the guidewire has a single-piece construction and comprises a tapered core mandrel with a stiffer proximal end and a flexible, shaped distal end. Such wires are often coated with a hydrophilic substance that increases lubricity on contract with blood. One example of this type of wire construction is the Glidewire® by Terumo of Japan. This type of wire is particularly useful for advancing through blood vessels that are blocked by thrombus or atherosclerosis.

In one embodiment, the guidewire has a multi-piece construction comprising a moveable inner core and an outer helical wound coil, with an opening at its proximal end and a closed-off distal end, creating a closed-tip lumen for the moveable core. In another embodiment, the distal tip is open-ended and the guidewire has a through-lumen that may be used for injecting or withdrawing diagnostic or therapeutic substances. The distal end of the coil may be preshaped into a “J”, “hockey-stick” or other configuration, or may contain a deformable inner strip or a shaping ribbon that allows the operator to create a desired tip configuration. In one embodiment, the core provides variable stiffness to at least a portion of the guidewire body. In one embodiment, the distal tip of the core may be tapered to create a smooth transition from the stiff portion to the flexible portion of the guidewire. In another embodiment, the tip may be rounded to improve passage of the core through the coil. In yet another embodiment, movement of the core may be facilitated with lubrication such as silicone oil or a polymeric coating. In one embodiment, the outer coil may be coated or bonded with a material such as Teflon to alter lubricity and/or an anticoagulant such as heparin. In one embodiment, the distal end of the core is capable of forming a friction fit or a mechanical interfit with the distal end of the coil with respect to rotation and facilitate the transmission of torque applied at the proximal core to the distal tip. This allows the user to alter the orientation of the distal tip and allow selection of vessels or other lumens as the wire is advanced and “torqued.” Moveable core guidewires may be advantageously used to position catheters in the body through a tortuous path while reducing trauma to body structures.

In another example of multi-piece construction, the core is fixed to a distal flexible coil that covers the distal tapered portion of the core transitioning into a shapeable tip. In one embodiment, such guidewires provide improved torque control. In another embodiment, the guidewire has a radio-opaque plating (such as a platinum or gold plating) applied to at least the distal end of the coil to aid in fluoroscopic visualization. In one embodiment, up to about 15 cm of the distal end is rendered radio-opaque. In a preferred embodiment, the distal 2 cm to 10 cm end of the coil is radio-opaque. These wires are used to selectively steer into small branches and provide a trackable path for interventional devices such as balloons or stents. Another variant of this type of construction is the wire described by Inoue as manufactured by the Toray Corporation of Japan. This wire has about a 0.025″ outer diameter stainless steel mandrel that tapers, with the distal portion covered by a flexible coil and configured in a spiral shape. This wire is particularly useful for securing a stable position in the left atrium after transseptal catheterization.

As shown in FIGS. 13A through 13C, in one embodiment, the guidewire 10 has an internal lumen 32. In one embodiment, the lumen 32 extends generally throughout the length of the guidewire. In another embodiment, the lumen 32 extends generally from about 10% to about 99% of the length of the guidewire. In still another embodiment, the lumen 32 generally extends about 95% of the guidewire length from the proximal end of the guidewire 10. In one embodiment, the lumen 32 has an internal diameter between about 0.012 inches to about 0.045 inches, preferably between about 0.020 inches to about 0.030 inches, and more preferably between about 0.020 inches to 0.025 inches. In one embodiment, the internal lumen 32 contains a core mandrel 33, shaft, and/or device for facilitating insertion and steerability of the guidewire 10. The core mandrel 33 has an outer diameter between about 0.012 inches to about 0.045 inches, preferably between about 0.020 inches to about 0.030 inches, and more preferably between about 0.020 to 0.025 inches. The core mandrel 33 has a length between about 20% to about 200% of the guidewire 10 length, preferably between about 50% to about 120%, and more preferably about 110%. The core mandrel may be moveable, removable, fixed or a combination thereof. By adjusting the position of a moveable or removable mandrel 33 within the guidewire 10, the stiffness of the guidewire may be adjusted by the user. In one embodiment, increased stiffness of the guidewire 10 may improve the steerability of the guidewire 10 to the target site and provide increased column strength to pass a device over the guidewire 10 without deforming the guidewire 10 and changing the insertion pathway or dislodging the distal portion of the guidewire 10 from the target site. By removing the mandrel 33, the flexibility of the guidewire 10 is increased to allow passage through tortuous routes in the body. In some embodiments of the invention, the distal portion 12 of the guidewire 10 is capable of coiling or assuming a preconfigured shape when the mandrel 33 is in the retracted position. In one embodiment, the distal portion 12 of the guidewire 10 forms a J-shape when the mandrel 33 is in the retracted position. In another embodiment, the guidewire 10 forms a coil shape. One skilled in the art will understand that the distal portion 12 of the guidewire 10 can be configured to provide steerability to and anchoring at any of a variety of target sites in the body, including but not limited to, the right atrium, left atrium, coronary sinus, pulmonary artery, left ventricle, aorta, stomach, duodenum, gallbladder, pancreas, renal calyxes, ureters, bladder and nasopharynx. In one embodiment, shown for example in FIG. 13A, the mandrel 33 is shown in a partially retracted position to allow flexibility in the distal portion 12 of the guidewire 10 and allows the inherent bias in the distal portion 12, if any, to assume a preconfigured shape, such as a coil or J-shape. In FIG. 13B, the mandrel 33 is in a fully extended position to generally stiffen the entire length of the guidewire 10 and to overcome at least some of the inherent bias of the distal portion 12 and at least partially straighten the distal portion 12. The mandrel 33 is capable of partial retraction and extension to vary the extent of the guidewire stiffening.

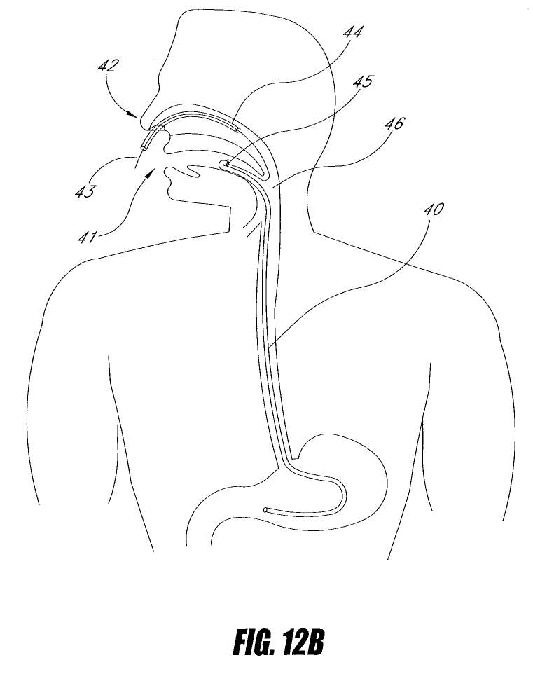

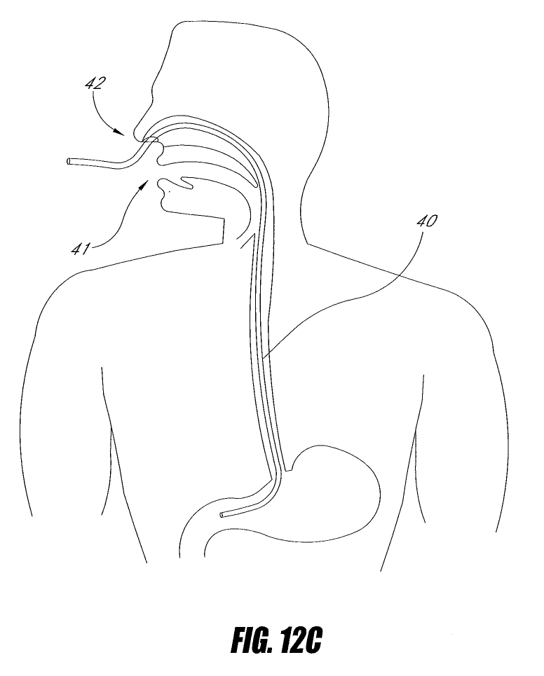

FIGS. 14A to 14C show another embodiment of the guidewire 10 comprising a distal fixed core 47 and a guidewire lumen 32 with moveable or removable core 33. In one embodiment, the guidewire lumen 32 has a proximal open end 48 and a closed distal end 49, with a length that is generally less than the full length of the guidewire 10. Preferably, the distal end 49 of the guidewire lumen 32 is positioned generally in the portion of the guidewire that transitions from the proximal straight portion to the preshaped distal portion 12. In one embodiment, the fixed distal core advantageously maintains the stiffness of preshaped distal portion 12 for anchoring the distal guidewire in the desired position, while the moveable core enhances flexibility during the repositioning of the proximal portion of the guidewire 10. In one embodiment, the distal fixed core comprises a stiff radio-opaque material, such as a platinum or gold alloy.

In one embodiment, the movable core mandrel 33 has a proximal end 35 with a tab 36 or other type of handle to facilitate manipulation of the mandrel 33. In another embodiment, the mandrel 33 lacks a tab 36 so that a device can be passed over guidewire 10 without having to remove mandrel 33. The movable core mandrel 33 may have a tapered distal end 37 to facilitate insertion and extension of the mandrel 33 through the internal lumen 32 of the guidewire 10. In one embodiment, the mandrel 33 is made from stainless steel or nickel titanium alloy (e.g., Nitinol). One skilled in the art will understand that the material and structure selected for the mandrel 33 can be based upon the desired stiffness, ductility, elastic deformation and other characteristics desired.

In one embodiment, the guidewire 10 is flexible or deformable, and the mandrel 33 is more rigid. In another embodiment, the mandrel 33 is flexible or deformable, and the guidewire 10 is more rigid. In one embodiment, the more rigid guidewire 10 comprises an opening at the distal end so that it can be passed over the proximal end of the mandrel and into the target site.

In one embodiment, the guidewire 10 is uniformly flexible along its length. In another embodiment, the pliancy of the guidewire 10 is not uniform throughout the length of the guidewire 10, even when the mandrel 33 is completely removed from the internal lumen 32. In a preferred embodiment, the middle portion of the guidewire 10 is more flexible than the distal end and/or the proximal end of the guidewire 10. One advantage of this alternating flexibility is that it facilitates bending and/or sharp turns in the body lumen.

In one embodiment, the guidewire comprises a material and structure with sufficient ductility capable of withstanding deformation of at least about 180 degrees to about 540 degrees of bending within a body or sheath lumen without breakage. In another embodiment, the guidewire comprises a material and structure with sufficient ductility and a yield point capable of withstanding deformation of at least about 220 degrees in a body or sheath lumen without breakage or plastic deformation. The guidewire may be made in whole or in part from a material selected from one or more of the following: stainless steel alloys such as NP35-N, nickel titanium (nitinol), tantalum, or a combination thereof. Similarly, the guidewire may be constructed from polymeric or composite materials including but not limited to polyethylenes, polyurethanes, carbon fibers, or blended combinations thereof. In another embodiment, the guidewire may be constructed of a combination of metallic and polymeric/composite materials. In another embodiment, the guidewire is coated with a hydrophilic coating or a polymer such as ePTFE to facilitate the passage of the guidewire through the body. One skilled in the art can select the guidewire material and structure to provide the desired characteristics, including but not limited to torqueability, stiffness, ductility, friction coefficient, radio-opacity and deformation characteristics.

While this invention has been particularly shown and described with references to embodiments thereof, it will be understood by those skilled in the art that various changes in form and details may be made therein without departing from the scope of the invention. For all of the embodiments described above, the steps of the methods need not be performed sequentially.

What is claimed is:

1. A device for manipulating an implantable lead member, comprising:
a biocompatible elongate member having a first section and a second section;
wherein the first section comprises a pull member attachment interface and the second section comprises a selective coupling interface configured to selectively attach to a section of an implantable lead member.

2. The device as in claim 1, wherein the pull member interface is a guidewire attachment interface.

3. The device as in claim 1, wherein the pull member interface is a snare attachment interface.

4. The device as in claim 1, wherein the pull member interface comprises a cavity configured to accept a pull member, a collet structure about the cavity, and a locking member configured to selectively compress the collet structure.

5. The device as in claim 1, wherein the selective coupling interface comprises a cavity configured to accept the implantable lead member, a collet structure about the cavity, and a locking member configured to selectively compress the collet structure.

6. The device as in claim 1, wherein the selective coupling interface comprises a catheter.

7. The device as in claim 1, wherein the selective coupling interface comprises a rotational coupling.

8. The device as in claim 1, wherein the biocompatible elongate member comprises a flexible material.

9. The device as in claim 1, wherein the biocompatible elongate member comprises a rigid material.

10. A transfer guidewire assembly configured to manipulate an implanted elongate body, the transfer guidewire assembly comprising:
a flexible elongate body, the flexible elongate body having a proximal end and a distal end; and
a coupler attached to the flexible elongate body's distal end,
wherein the coupler is configured to be removably attached to the end of an implanted elongate body.

11. The transfer guidewire assembly of claim 10, wherein the flexible elongate body comprises a guidewire.

12. The transfer guidewire assembly of claim 10, wherein the coupler comprises a screw.

13. The transfer guidewire assembly of claim 10, wherein the coupler is configured to rotate about the flexible elongate body.

14. The transfer guidewire assembly of claim 10, wherein the coupler is configured to prevent rotational forces from acting upon the implanted elongate body as the flexible elongate body is withdrawn from the patient's body while attached to the implanted elongate body.

15. The transfer guidewire assembly of claim 10, further comprising a stylet extending from the distal end of the flexible elongate body.

16. The transfer guidewire assembly of claim 10, wherein the coupler comprises a rotational coupling.

17. The transfer guidewire assembly of claim 10, wherein the implanted elongate body comprises a sensor lead.

18. A transfer guidewire assembly configured to reposition an end of an implanted lead from a first access point at a patient's body to a second access point at the patient's body, the transfer guidewire assembly comprising:
a flexible guidewire, the flexible guidewire having a proximal end and a distal end; and
a rotational coupling attached to the guidewire's distal end,
wherein the rotational coupling is configured to removably attach to a lead implanted within a patient's body.

19. The transfer guidewire assembly of claim 18, wherein the rotational coupling comprises a housing having an atraumatic surface configured to be pulled through the patient's body from a first access point to a second access point while attached to the implanted lead without damaging tissue within the patient's body

20. The transfer guidewire assembly of claim 18, wherein the rotational coupling comprises a screw configured to mate with the implanted lead.

21. The transfer guidewire assembly of claim 18, further comprising a stylet extending from a distal end of the rotational coupling and sized to enter the implanted lead.

22. A transfer guidewire assembly configured to reposition an end of an implantable, flexible, elongate body from a first access point at a patient's body to a second access point at the patient's body, the transfer guidewire assembly comprising:
a flexible guidewire, the flexible guidewire having a proximal end and a distal end; and
a catheter attached to the guidewire's proximal end,
wherein the catheter is configured to be removably coupled to an end of a flexible, elongate body implantable within a medical patient.

23. The transfer guidewire assembly of claim 22, wherein the distal end of the flexible guidewire is retrievable with a snare.

24. The transfer guidewire assembly of claim 22, wherein the distal end of the flexible guidewire comprises a J-tip.

25. The transfer guidewire assembly of claim 22, further comprising a stylet configured to be inserted into the implantable, flexible, elongate body.

26. The transfer guidewire assembly of claim 25, wherein the stylet comprises an elongate shaft of nickel titanium.

27. The transfer guidewire assembly of claim 25, wherein the stylet is integrally formed with the catheter and guidewire.

28. The transfer guidewire assembly of claim 25, wherein the stylet is wrapped around a proximal portion of the guidewire.

29. The transfer guidewire assembly of claim 28, wherein the wrapped proximal portion of the guidewire is surrounded by an end region of the catheter.

30. The transfer guidewire assembly of claim 25, wherein the stylet extends through and exits from a lumen formed by the catheter.

31. The transfer guidewire assembly of claim 22, wherein the catheter is configured to form an interference fit over the end of the flexible, elongate body.

32. The transfer guidewire assembly of claim 31, wherein the interference fit allows the catheter to remain attached to the implantable, flexible, elongate body when at least 1.5-times a rated pull force is exerted upon the catheter.
